(19) United States
(12) Patent Application Publication
(10) Pub. No.: US 2021/0007709 A1
(43) Pub. Date: Jan. 14, 2021

(54) MEASUREMENT APPARATUS, ULTRASOUND DIAGNOSTIC APPARATUS, MEASUREMENT METHOD, AND MEASUREMENT PROGRAM

(71) Applicant: FUJIFILM Corporation, Tokyo (JP)

(72) Inventors: Hisashi ENDO, Kanagawa (JP); Masayuki TAKAHIRA, Kanagawa (JP)

(21) Appl. No.: 16/891,322

(22) Filed: Jun. 3, 2020

(30) Foreign Application Priority Data
Jul. 10, 2019 (JP) 2019-128479

(51) Int. Cl. A61B 8/12; G02B 23/24; A61B 8/00

(52) U.S. Cl. CPC A61B 8/12; A61B 8/4494; G02B 23/2423

(57) ABSTRACT

A measurement controller generates a first brightness profile on a first straight line passing through a first measurement point and a second measurement point specified via a console with respect to an ultrasound image displayed on the monitor, detects a measurement candidate point that is a correction candidate of a position of at least one of the first measurement point or the second measurement point on the first straight line on the basis of the first brightness profile, and displays the measurement candidate point on the monitor. For example, in a case where a set button is pressed in the state of FIG. 8, the first measurement point is determined as a first final measurement point, the second measurement candidate point is determined as a second final measurement point, and a distance between the first final measurement point and the second final measurement point is measured.

### CROSS-REFERENCE TO RELATED APPLICATIONS

The present application claims priority under 35 U.S.C. § 119 to Japanese Patent Application No. 2019-128479 filed on Jul. 10, 2019. Each of the above application is hereby expressly incorporated by reference, in its entirety, into the present application.

### BACKGROUND OF THE INVENTION

### 1. Field of the Invention

The present invention relates to a measurement apparatus, an ultrasound diagnostic apparatus, a measurement method, and a measurement program.

### 2. Description of the Related Art

An ultrasound diagnostic apparatus that acquires an ultrasound image of the inside of a subject by driving each of a plurality of ultrasound transducers inside the subject (for example, the body of a patient) to transmit and receive ultrasound waves is already known. An ultrasound diagnostic apparatus having a measurement function of measuring the size of tissue included in the ultrasound image (for example, see JP2005-334089A, JP2019-083960A, and JP2008-161220A) is known.

JP2005-334089A and JP2019-083960A disclose an ultrasound diagnostic apparatus that specifies a plurality of measurement points on an ultrasound image displayed on a display unit using an input apparatus such as keyboards and trackballs and measures a range specified by the plurality of measurement points.

JP2008-161220A discloses an ultrasound diagnostic apparatus that generates a brightness profile on a detection line by setting the detection line for an ultrasound image displayed on a display unit, sets two measurement points on the detection line on the basis of the brightness profile, and measures a composite thickness of the intima and media of a blood vessel on the basis of the two measurement points.

### SUMMARY OF THE INVENTION

The measurement function in the ultrasound diagnostic apparatus is often performed while a subject is being tested. In order to specify a measurement range on the ultrasound image, it is necessary to set at least two measurement points. However, it is not easy to accurately specify the measurement range, in other words, to accurately specify a position of the measurement point within a limited time during the test.

In JP2005-334089A and JP2019-083960A, since the measurement is performed under the assumption that a measurement point specified by a manual operation is correct, the reliability of a measurement result can be reduced. JP2008-161220A determines a measurement range on the basis of the brightness profile on the detection line. However, in this method, since only the position of the detection line can be specified, the measurement of tissues different from the desired tissue can be performed. Also, depending on the state of the brightness profile, measurement of tissues different from the desired tissue can be performed.

The present invention has been accomplished in consideration of the above-described situation, and an object of the invention is to provide a measurement apparatus, an ultrasound diagnostic apparatus, a measurement method, and a measurement program that can set an intended measurement target range with high accuracy for an ultrasound image.

A measurement apparatus according to the aspect of the present invention comprises a correction support information generation unit that generates a first brightness profile on a first straight line passing through a first measurement point and a second measurement point specified for an ultrasound image generated by an ultrasound diagnostic apparatus and displayed on a display unit, and generates first correction support information for supporting correction of at least one position of the first measurement point or the second measurement point on the first straight line on the basis of the first brightness profile, and a measurement unit that displays the first correction support information on the display unit, and on the basis of instructions input in a state where the first correction support information is displayed on the display unit, determines one of the first measurement point or a first corrected measurement point corrected from the first measurement point based on the first correction support information as a first final measurement point, determines one of the second measurement point or a second corrected measurement point corrected from the second measurement point based on the first correction support information as a second final measurement point, and measures a size of a measurement range on the ultrasound image based on the first final measurement point and the second final measurement point.

An ultrasound diagnostic apparatus according to the aspect of the present invention comprises the measurement apparatus and an image processing unit that generates the ultrasound image on the basis of an output signal of an ultrasonic endoscope.

The measurement method according to the aspect of the present invention comprises a correction support information generation step of generating a first brightness profile on a first straight line passing through a first measurement point and a second measurement point specified for an ultrasound image generated by an ultrasound diagnostic apparatus and displayed on a display unit, and generating first correction support information for supporting correction of at least one position of the first measurement point or the second measurement point on the first straight line on the basis of the first brightness profile, and a measurement step of displaying the first correction support information on the display unit, and on the basis of instructions input in a state where the first correction support information is displayed on the display unit, determining one of the first measurement point or a first corrected measurement point corrected from the first measurement point based on the first correction support information as a first final measurement point, determining one of the second measurement point or a second corrected measurement point corrected from the second measurement point based on the first correction support information as a second final measurement point, and measuring a size of a measurement range on the ultrasound image based on the first final measurement point and the second final measurement point.

The measurement program according to the aspect of the present invention is a program for causing a computer to perform a correction support information generation step of generating a first brightness profile on a first straight line passing through a first measurement point and a second measurement point specified for an ultrasound image generated by an ultrasound diagnostic apparatus and displayed on a display unit, and generating first correction support information for supporting correction of at least one position of the first measurement point or the second measurement point on the first straight line on the basis of the first brightness profile, and a measurement step of displaying the first correction support information on the display unit, and on the basis of instructions input in a state where the first correction support information is displayed on the display unit, determining one of the first measurement point or a first corrected measurement point corrected from the first measurement point based on the first correction support information as a first final measurement point, determining one of the second measurement point or a second corrected measurement point corrected from the second measurement point based on the first correction support information as a second final measurement point, and measuring a size of a measurement range on the ultrasound image based on the first final measurement point and the second final measurement point.

According to the present invention, it is possible to provide a measurement apparatus, an ultrasound diagnostic apparatus, a measurement method, and a measurement program that can set a measurement target range intended for an ultrasound medical image with high accuracy.

### BRIEF DESCRIPTION OF THE DRAWINGS

FIG. 1 is a diagram illustrating a schematic configuration of an ultrasonic endoscope apparatus 10.

FIG. 2 is a block diagram illustrating a configuration of an ultrasonic endoscope 12 and an ultrasonic processor apparatus 14.

FIG. 3 is a schematic diagram illustrating an external configuration of a console 100.

FIG. 4 is a diagram illustrating a functional block of a measurement controller 158.

FIG. 5 is a schematic diagram illustrating an example of a screen displayed on a monitor 20 in a distance measurement mode.

FIG. 6 is a diagram illustrating an example of a first brightness profile of a first straight line L1 illustrated in FIG. 5.

FIG. 7 is a schematic diagram illustrating a state where a correction candidate of a second measurement point is additionally displayed on the screen illustrated in FIG. 5.

FIG. 8 is a schematic diagram illustrating a state where correction candidates of a first measurement point and a second measurement point are additionally displayed on the screen illustrated in FIG. 5.

FIG. 9 is a schematic diagram illustrating an example of a screen displayed in a case where a determination operation (pressing a set button) of a measurement point is performed from the state of FIG. 7.

FIG. 10 is a schematic diagram illustrating another example of the screen displayed on the monitor 20 in the distance measurement mode.

FIG. 11 is a diagram illustrating an example of a first brightness profile of a first straight line L1 illustrated in FIG. 10.

FIG. 12 is a schematic diagram illustrating an example of a screen displayed on the monitor 20 as a result of analyzing the first brightness profile illustrated in FIG. 11.

FIG. 13 is a schematic diagram illustrating an example of a screen displayed on the monitor 20 in a case where a set button 104 is pressed on the screen illustrated in FIG. 12.

FIG. 14 is a schematic diagram illustrating a state where the first brightness profile illustrated in FIG. 11 and information indicating a position of the first measurement point A and the second measurement point B in the first brightness profile are additionally displayed on the monitor 20 with respect to the screen of FIG. 10.

FIG. 15 is a schematic diagram illustrating a state where two points on a graph PF in a subsidiary screen G2 are selected on the screen illustrated in FIG. 14 and a set button 104 is pressed.

FIG. 16 is a schematic diagram illustrating an example of a screen displayed on a monitor 20 in an area measurement mode.

FIG. 17 is a schematic diagram illustrating a state where correction candidates of a second measurement point and a fourth measurement point are additionally displayed on the screen illustrated in FIG. 16.

FIG. 18 is a schematic diagram illustrating an example of a screen displayed in a case where a determination operation (pressing a set button) of a measurement point is performed from the state of FIG. 17.

FIG. 19 is a schematic diagram illustrating an example of a screen displayed on the monitor 20 in the area measurement mode.

FIG. 20 is a schematic diagram illustrating an example of a screen displayed on the monitor 20 in the area measurement mode.

### DESCRIPTION OF THE PREFERRED EMBODIMENTS

Outline of Ultrasound Diagnostic Apparatus

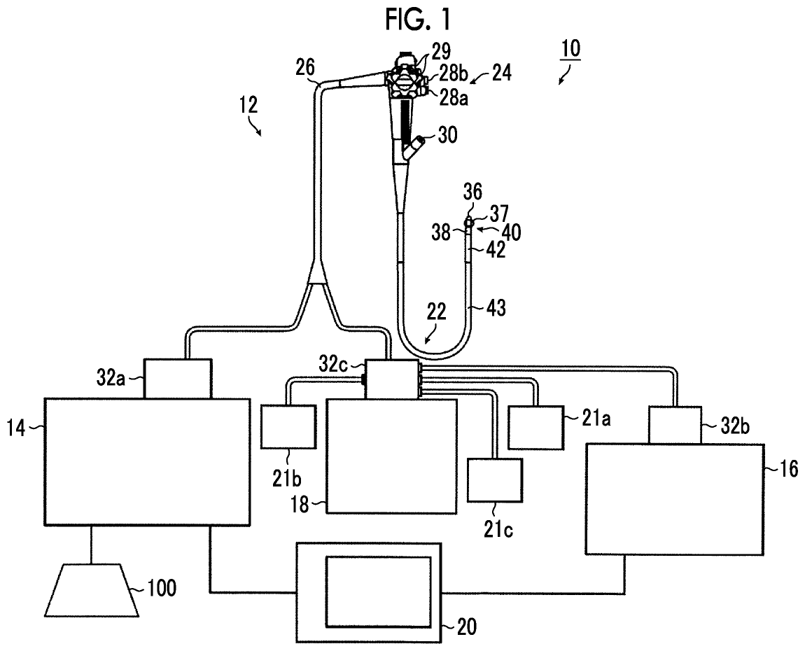

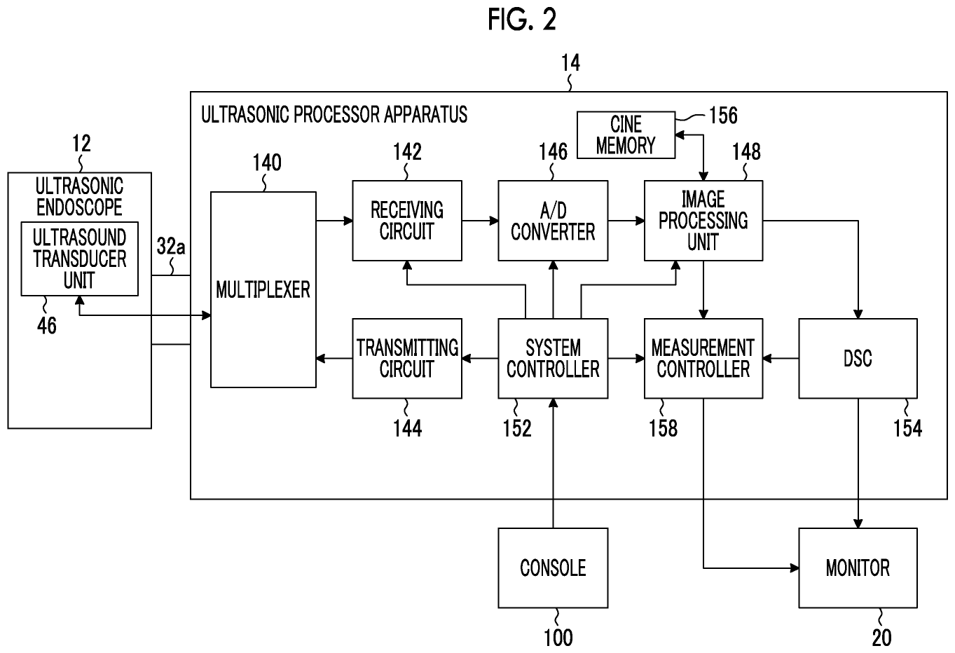

An outline of an ultrasonic endoscope apparatus 10 which is an embodiment of an ultrasound diagnostic apparatus according to the embodiment of the present invention will be described with reference to FIGS. 1 and 2. FIG. 1 is a diagram illustrating a schematic configuration of an ultrasonic endoscope apparatus 10. FIG. 2 is a block diagram illustrating a configuration of an ultrasonic endoscope 12 and an ultrasonic processor apparatus 14.

The ultrasonic endoscope apparatus 10 is used for observing the state of an observation target site in the body of a patient who is a subject (hereinafter, also referred to as an ultrasound diagnosis) using ultrasound waves. Here, the observation target site is a site that is difficult to test from the body surface side (outside) of the patient, such as the gall bladder or the pancreas. It is possible to perform the ultrasound diagnosis for the state of the observation target site and the presence or absence of abnormality via digestive tracts such as the esophagus, stomach, duodenum, small intestine, and large intestine, which are a body cavity of the patient, by using the ultrasonic endoscope apparatus 10.

As illustrated in FIG. 1, the ultrasonic endoscope apparatus 10 has an ultrasonic endoscope 12, an ultrasonic processor apparatus 14, an endoscopic processor apparatus 16, a light source apparatus 18, a monitor 20 forming a display unit, and a console 100 forming an operation unit. As illustrated in FIG. 1, a water supply tank 21a, a suction pump 21b, and an air supply pump 21c are provided as accessories of the ultrasonic endoscope apparatus 10. Further, a pipe line (not illustrated) serving as a flow path of water and gas is formed in the ultrasonic endoscope 12. The ultrasonic processor apparatus 14, the endoscopic processor apparatus 16, and the light source apparatus 18 constitute a main body unit of the ultrasonic endoscope apparatus 10.

As illustrated in FIG. 1, the ultrasonic endoscope 12 has an insertion part 22 inserted into the body cavity of a patient and an operation unit 24 operated by an operator (user) such as a doctor or a technician. In addition, an ultrasound transducer unit 46 comprising a plurality of ultrasound transducers is attached to a distal end part 40 of the insertion part 22.

The operator can acquire an endoscopic image of an inner wall of the body cavity of the patient and an ultrasound image of an observation target site by the function of the ultrasonic endoscope 12. The endoscopic image is an image obtained by imaging the inner wall of the body cavity of the patient by an optical method. The ultrasound image is an image obtained by receiving reflected waves (echo) of ultrasound waves transmitted from the body cavity of the patient toward an observation target site and imaging the received signal.

The ultrasonic processor apparatus 14 is connected to the ultrasonic endoscope 12 via a universal cord 26 and an ultrasonic connector 32a provided at an end part thereof. The ultrasonic processor apparatus 14 controls the ultrasound transducer unit 46 of the ultrasonic endoscope 12 to cause the ultrasound transducer unit 46 to transmit ultrasound waves. In addition, the ultrasonic processor apparatus 14 generates the ultrasound image by imaging the received signal in a case where the ultrasound transducer unit 46 receives the reflected waves (echo) of the ultrasound waves.

As illustrated in FIG. 1, the endoscopic processor apparatus 16 is connected to the ultrasonic endoscope 12 via the universal cord 26 and an endoscopic connector 32b provided at an end part thereof. The endoscopic processor apparatus 16 acquires image data of an observation target adjacent part picked-up by the ultrasonic endoscope 12, performs predetermined image processing on the acquired image data, and generates the endoscopic image.

As illustrated in FIG. 1, the light source apparatus 18 is connected to the ultrasonic endoscope 12 via the universal cord 26 and a light source connector 32c provided at an end part thereof. The light source apparatus 18 irradiates white light or specific wavelength light consisting of three primary colors of red light, green light and blue light in the case of imaging the observation target adjacent part using the ultrasonic endoscope 12. The light irradiated by the light source apparatus 18 propagates the ultrasonic endoscope 12 through a light guide (not illustrated) included in the universal cord 26, and is emitted from the ultrasonic endoscope 12. Additionally, the observation target adjacent part is irradiated by the light from the light source apparatus 18.

In the present embodiment, the ultrasonic processor apparatus 14 and the endoscopic processor apparatus 16 are configured by two apparatus (computers) separately provided. However, the present invention is not limited to this, and both the ultrasonic processor apparatus 14 and the endoscopic processor apparatus 16 may be configured by one apparatus.

The monitor 20 is connected to the ultrasonic processor apparatus 14 and the endoscopic processor apparatus 16, and displays an ultrasound image generated by the ultrasonic processor apparatus 14, an endoscopic image generated by the endoscopic processor apparatus 16, and the like. Regarding the display of the ultrasound image and the endoscopic image, either one of the images may be switched and displayed on the monitor 20, or both images may be displayed simultaneously. In addition, a configuration in which the display methods can be randomly selected and changed may be employed.

In the present embodiment, the ultrasound image and the endoscopic image are displayed on one monitor 20, but a monitor for displaying an ultrasound image and a monitor for displaying an endoscopic image may be separately provided. In addition, a display method other than the monitor 20, for example, a method in which an ultrasound image and an endoscopic image are displayed on a display of a personal terminal carried by an operator may be used.

The console 100 is an input apparatus provided for the operator to input necessary information for the ultrasound diagnosis or to instruct the ultrasonic processor apparatus 14 to start the ultrasound diagnosis, or the like. The console 100 is configured by, for example, a keyboard, a mouse, a trackball, a touch pad, a touch panel, or the like, or a combination thereof, and is connected to a system controller 152 of the ultrasonic processor apparatus 14 as illustrated in FIG. 2. In a case where the console 100 is operated, the system controller 152 of the ultrasonic processor apparatus 14 controls each unit of apparatus (for example, a later-described receiving circuit 142 and transmitting circuit 144) according to the operation content.

Further, the operator can set various control parameters on the console 100 in a case of performing the ultrasound diagnosis. The control parameters include, for example, a selection result of a live mode and a freeze mode, a set value of a display depth (depth), and a selection result of an ultrasound image generation mode.

Here, the “live mode” is a mode in which ultrasound images (motion pictures) obtained at a predetermined frame rate are sequentially displayed (real-time display). The “freeze mode” is a mode in which an ultrasound image (still pictures) for one frame acquired in the past is read out from a cine memory (not illustrated) and displayed.

There is a plurality of ultrasound image generation modes that can be selected in the present embodiment, and specifically, a B (brightness) mode, a CF (color flow) mode, and a PW (pulse wave) mode. The B mode is a mode in which an amplitude of an ultrasound echo is converted into brightness and a tomographic image is displayed. The CF mode is a mode in which an average blood flow velocity, a flow fluctuation, a flow signal intensity, a flow power, or the like are mapped to various colors and displayed in the B mode image in an overlapping manner. The PW mode is a mode in which speed (for example, blood flow velocity) of an ultrasound echo source detected on the basis of transmission and reception of a pulse wave is displayed. The above-described ultrasound image generation mode is merely an example, and modes other than the three types of modes described above, for example, an A (Amplitude) mode, an M (Motion) mode, and the like may be further included.

Configuration of Ultrasonic Endoscope

The ultrasonic endoscope 12 has an insertion part 22 and an operation unit 24. The insertion part 22 comprises a distal end part 40, a bending part 42, and a flexible part 43 in order from the distal end side (free end side). An ultrasonic observation part 36 and an endoscopic observation part 38 are provided at the distal end part 40.

A balloon 37 that is expandable and contractible is attached to the distal end part 40 at a position covering the ultrasound transducer unit 46.

The bending part 42 is a part provided closer to the base end side (the side opposite to the side where the ultrasound transducer unit 46 is provided) than the distal end part 40 of the insertion part 22, and is freely bendable. The flexible part 43 is a part that connects the bending part 42 and the operation unit 24, has flexibility, and is provided in an elongated state.

As illustrated in FIG. 1, the operation unit 24 is provided with a pair of angle knobs 29 and a treatment instrument insertion port 30. In a case where each angle knob 29 is moved rotationally, the bending part 42 is remotely operated to be bent and be deformed. The distal end part 40 of the insertion part 22 provided with the ultrasonic observation part 36 and the endoscopic observation part 38 can be directed in the desired direction by the deformation operation. The treatment instrument insertion port 30 is a hole formed for inserting a treatment instrument such as forceps, and connects with a treatment instrument lead-out port provided at the distal end part 40 via a treatment instrument channel.

The operation unit 24 is provided with an air and water supply button 28a for opening or closing an air and water supply pipe line (not illustrated) extending from the water supply tank 21a, and a suction button 28b for opening or closing a suction pipe line (not illustrated) extending from the suction pump 21b.

The other end part of the universal cord 26 is provided with the ultrasonic connector 32a connected to the ultrasonic processor apparatus 14, the endoscopic connector 32b connected to the endoscopic processor apparatus 16, and the light source connector 32c connected to the light source apparatus 18. The ultrasonic endoscope 12 is attachably and detachably connected to the ultrasonic processor apparatus 14, the endoscopic processor apparatus 16, and the light source apparatus 18 via connectors 32a, 32b, and 32c, respectively.

Next, the ultrasonic observation part 36 among components of the ultrasonic endoscope 12 will be described.

Ultrasonic Observation Part

The ultrasonic observation part 36 is a part provided for acquiring an ultrasound image, and is disposed on the distal end side of the distal end part 40 of the insertion part 22. The ultrasonic observation part 36 comprises the ultrasound transducer unit 46 illustrated in FIG. 2. The ultrasound transducer unit 46 is a convex probe in which N (N is 2 or more) ultrasound transducers are arranged in a circular-arc shape, and transmits ultrasound waves in a radial shape (circular-arc shape). The type (model) of the ultrasound transducer unit 46 is not particularly limited, and may be another type as long as it can transmit and receive ultrasound waves, for example, a sector type, a linear type, a radial type, and the like.

Each ultrasound transducer of the ultrasonic observation part 36 is supplied with a pulsed driving voltage from the ultrasonic processor apparatus 14 as an input signal. In a case where the driving voltage is applied to an electrode of the ultrasound transducer, a piezoelectric element expands and contracts, and the ultrasound transducer is driven (vibrated). As a result, pulsed ultrasound waves are output from the ultrasound transducer. In addition, in a case where reflected waves (echo) of the ultrasound waves or the like are received, each ultrasound transducer vibrates (drives) accordingly, and the piezoelectric element of each ultrasound transducer generates an electric signal. The electric signal is output from each ultrasound transducer toward the ultrasonic processor apparatus 14 as a received signal.

The ultrasound transducer unit 46 of the present embodiment has a convex type as described above. That is, in the present embodiment, the ultrasound waves are scanned in a scanning range along a curved surface, for example, in a range of about several tens mm from the center of curvature of the curved surface by sequentially driving the N ultrasound transducers included in the ultrasound transducer unit 46 by an electronic switch such as a multiplexer 140 described later.

Configuration of Ultrasonic Processor Apparatus

As illustrated in FIG. 2, the ultrasonic processor apparatus 14 has a multiplexer 140, a receiving circuit 142, a transmitting circuit 144, an A/D (Analog Digital) converter 146, an image processing unit 148, a system controller 152, a digital scan converter (DSC) 154, a cine memory 156 and a measurement controller 158.

The receiving circuit 142 and the transmitting circuit 144 are electrically connected to each ultrasound transducer of the ultrasonic endoscope 12 via the multiplexer 140. The multiplexer 140 selects one or more from N ultrasound transducers (N is a natural number of 2 or more) and opens the channel.

The transmitting circuit 144 is a circuit that supplies a driving voltage for transmitting ultrasound waves to the ultrasound transducer selected by the multiplexer 140 in order to transmit the ultrasound waves from the ultrasound transducer unit 46.

The receiving circuit 142 is a circuit that receives an electric signal output from the ultrasound transducer received the ultrasound waves (echo), that is, a received signal. In addition, the receiving circuit 142 amplifies the received signal received from the ultrasound transducer according to a control signal sent from the system controller 152, and delivers the amplified signal to the A/D converter 146. The A/D converter 146 is connected to the receiving circuit 142, converts the received signal received from the receiving circuit 142 from an analog signal to a digital signal, and outputs the converted digital signal to the image processing unit 148.

The image processing unit 148 generates an ultrasound image on the basis of the digital received signal output from the A/D converter 146. The ultrasound image generated by the image processing unit 148 is stored in the cine memory 156. In a case where an operation of reading out an ultrasound image is performed by the console 100, the image processing unit 148 reads out the ultrasound image specified from the cine memory 156 and transfers the image to a DSC 154.

The DSC 154 converts (raster-converts) a signal of the ultrasound image (including the image read out from the cine memory 156) generated by the image processing unit 148 into an image signal according to a normal television signal scanning method, performs various necessary image processing such as gradation processing on the image signal, and outputs the image signal to the monitor 20.

The system controller 152 controls each unit of the ultrasonic processor apparatus 14, and is connected to the receiving circuit 142, the transmitting circuit 144, the A/D converter 146, the image processing unit 148, and the measurement controller 158, to control the apparatus. The system controller 152 is connected to the console 100, and controls each unit of the ultrasonic processor apparatus 14 according to test information and control parameters input at the console 100 in a case where the subject is tested. Thereby, an ultrasound image according to the ultrasound image generation mode specified by the operator is acquired.

The measurement controller 158 measures the size (length, area, or the like) of a measurement range specified via the console 100 in the ultrasound image displayed on the monitor 20, and displays a measurement result on the monitor 20. At the time of specifying the measurement range, the measurement controller 158 also performs control to support this. The measurement controller 158 constitutes a measurement apparatus.

Each of the image processing unit 148, the system controller 152, and the measurement controller 158 includes various processors that execute programs to perform processing, a random access memory (RAM), and a read only memory (ROM).

The various processors in the embodiment of the present invention include a central processing unit (CPU) which is a general-purpose processor that executes programs to perform various processing, a programmable logic device (PLD) which is a processor whose a circuit configuration can be changed after manufacturing such as a field programmable gate array (FPGA), or a dedicated electric circuit which is a processor having a circuit configuration specifically designed to execute specific processing such as an application specific integrated circuit (ASIC). More specifically, the structures of the various processors are electric circuits in which circuit elements such as semiconductor elements are combined.

The system controller 152 may be configured with one of various processors, or configured with a combination of two or more processors of the same type or different types (for example, a combination of a plurality of FPGAs or a combination of a CPU and an FPGA).

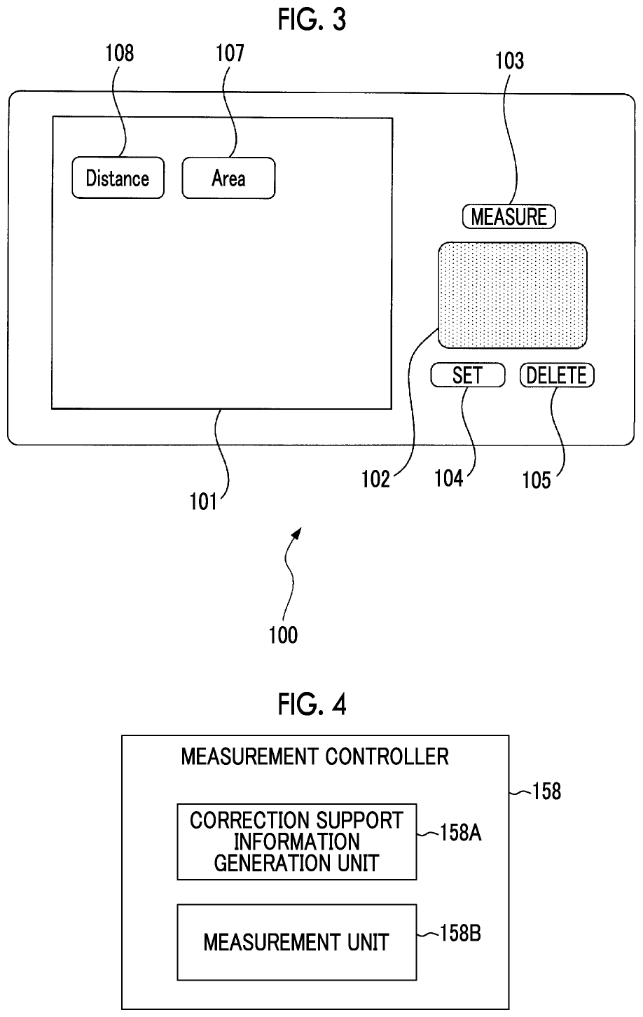

FIG. 3 is a schematic diagram illustrating an external configuration of a console 100. The console 100 comprises a touch panel 101 integrated with a display apparatus such as a liquid crystal display, a touch pad 102, a measure button 103 for instructing start of a measurement mode, a set button 104, and a delete button 105. A track ball or a touch panel may be provided instead of the touch pad 102.

FIG. 3 illustrates a state where the measure button 103 is pressed while an ultrasound image is displayed on the monitor 20. A distance button 108 that instructs the start of a distance measurement mode for measuring a distance between two points specified on the ultrasound image and an area button 107 that instructs the start of an area measurement mode for measuring an area of an elliptical range specified on an ultrasound image are displayed on the touch panel 101. In a case where the console 100 is operated in the distance measurement mode and the area measurement mode, a signal corresponding to the operation is transmitted to the measurement controller 158 via the system controller 152.

FIG. 4 is a diagram illustrating a functional block of a measurement controller 158. The processor of the measurement controller 158 functions as the measurement apparatus comprising a correction support information generation unit 158A and a measurement unit 158B by executing a measurement program.

The correction support information generation unit 158A generates a first brightness profile on a first straight line passing through a first measurement point and a second measurement point specified via the console 100 with respect to the ultrasound image displayed on the monitor 20 in the distance measurement mode, and generates first correction support information for supporting correction of a position of at least one of the first measurement point or the second measurement point on the first straight line, on the basis of the first brightness profile.

The correction support information generation unit 158A generates second correction support information in addition to the first correction support information in the area measurement mode. Specifically, the correction support information generation unit 158A generates a second brightness profile on a second straight line passing through a third measurement point and a fourth measurement point specified via the console 100 with respect to the ultrasound image displayed on the monitor 20 and orthogonal to the first straight line, and generates the above-described second correction support information for supporting correction of a position of at least one of the third measurement point or the fourth measurement point on the second straight line, on the basis of the second brightness profile.

The measurement unit 158B causes the monitor 20 to display the first correction support information generated by the correction support information generation unit 158A in the distance measurement mode. In this state, the measurement unit 158B measures a distance between two points (specifically, any one of the first measurement point or a first corrected measurement point corrected from the first measurement point based on the first correction support information displayed on the monitor 20 and any one of the second measurement point or a second corrected measurement point corrected from the second measurement point based on the first correction support information displayed on the monitor 20) determined via the console 100 on the ultrasound image displayed on the monitor 20, and displays the measurement result on the monitor 20.

The measurement unit 158B causes the monitor 20 to display the first correction support information and the second correction support information generated by the correction support information generation unit 158A in the area measurement mode. In this state, the measurement unit 158B measures the area of the elliptical range determined on the basis of the four points (specifically, any one of the first measurement point or a first corrected measurement point corrected from the first measurement point based on the first correction support information displayed on the monitor 20, any one of the second measurement point or a second corrected measurement point corrected from the second measurement point based on the first correction support information displayed on the monitor 20, any one of the third measurement point or a third corrected measurement point corrected from the third measurement point based on the second correction support information displayed on the monitor 20, and any one of the fourth measurement point or a fourth corrected measurement point corrected from the fourth measurement point based on the second correction support information displayed on the monitor 20) determined via the console 100 on the ultrasound image displayed on the monitor 20, and displays the measurement result on the monitor 20.

The measurement controller 158 operates in the distance measurement mode in a case where the measure button 103 illustrated in FIG. 3 is pressed and the distance button 108 is pressed. In a case where the measure button 103 illustrated in FIG. 3 is pressed and the area button 107 is pressed, the measurement controller 158 operates in the area measurement mode. Hereinafter, the operation in each mode will be described in detail.

Operation in Distance Measurement Mode

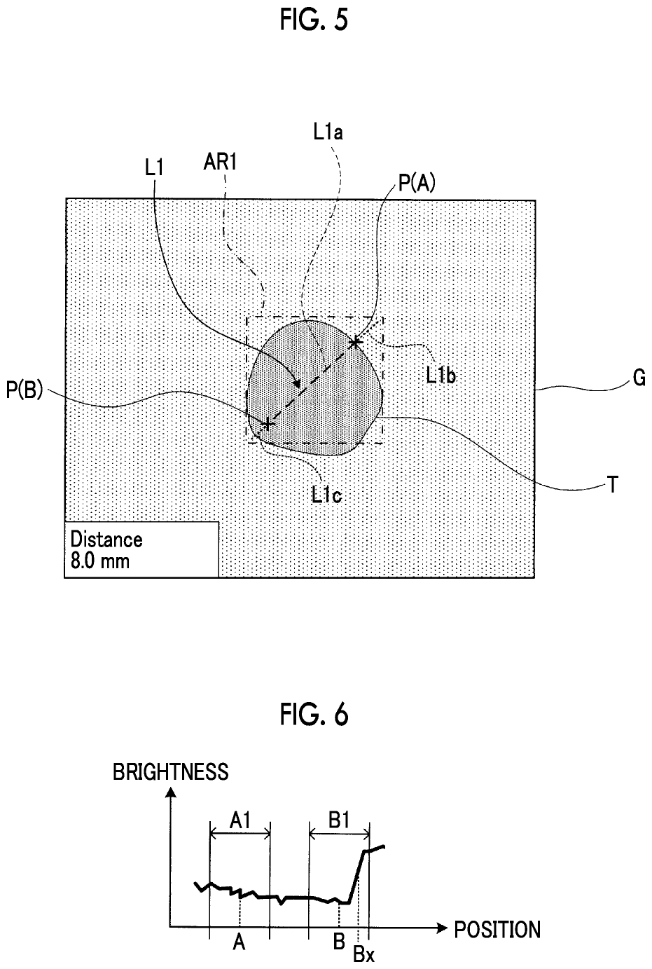

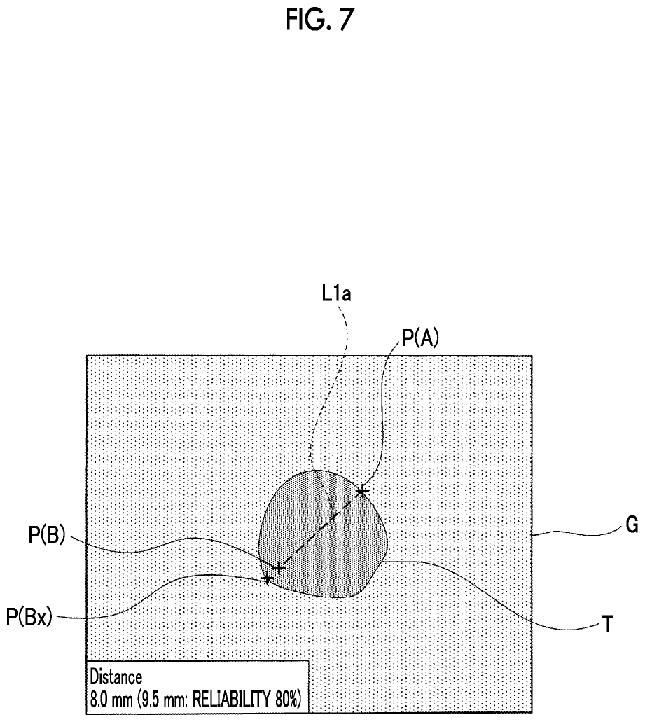

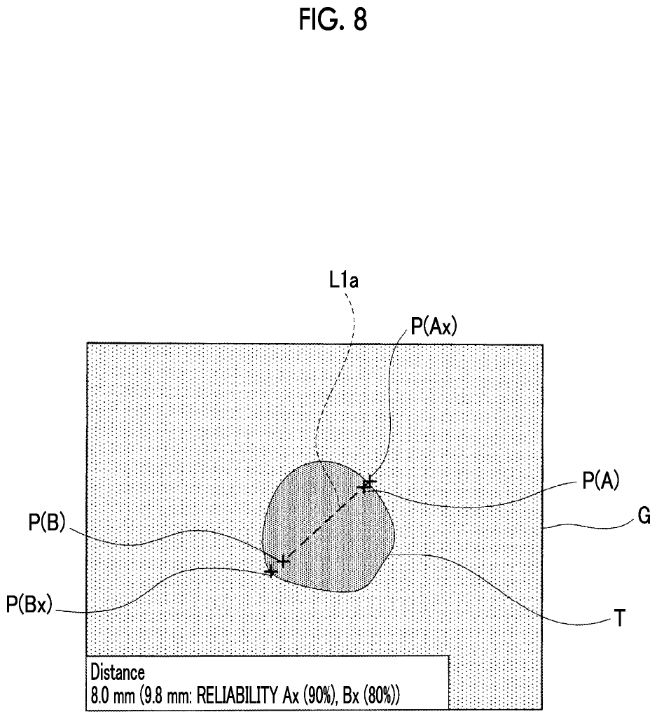

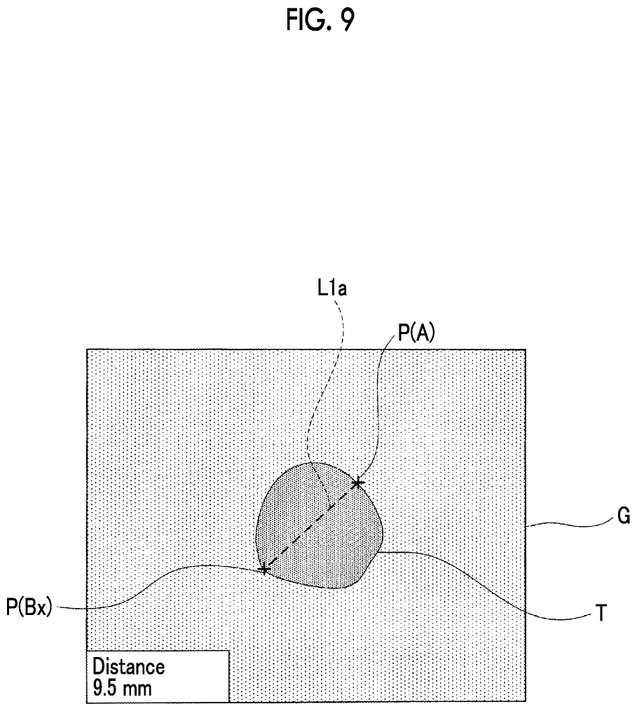

FIG. 5 is a schematic diagram illustrating an example of a screen displayed on a monitor 20 in a distance measurement mode. FIG. 6 is a diagram illustrating an example of a first brightness profile of a first straight line L1 illustrated in FIG. 5. FIG. 7 is a schematic diagram illustrating a state where a correction candidate of a second measurement point is additionally displayed on the screen illustrated in FIG. 5. FIG. 8 is a schematic diagram illustrating a state where correction candidates of a first measurement point and a second measurement point are additionally displayed on the screen illustrated in FIG. 5. FIG. 9 is a schematic diagram illustrating an example of a screen displayed in a case where a determination operation (pressing a set button) of a measurement point is performed from the state of FIG. 7.

In a case where the operator operates the console 100 and gives an instruction to display an ultrasound image acquired in the B mode stored in the cine memory 156, the DSC 154 causes the monitor 20 to display the ultrasound image. In addition, the measurement controller 158 acquires the ultrasound image displayed on the monitor 20 by the DSC 154. Then, the measurement unit 158B displays a pointer P for specifying a measurement point at a random position on the ultrasound image being displayed on the monitor 20. The position of the pointer P displayed on the monitor 20 is changed by operating the touch pad 102. In a case where the set button 104 is pressed while the pointer P is at the random position, the measurement unit 158B receives an instruction to specify the position as a measurement point, causes the pointer P to be fixedly displayed on the position, and stores position information (coordinates) of the pointer P. Specifying the measurement point described here may be performed using an input apparatus other than the console 100. As such an input apparatus, for example, the operation unit 24 provided in the ultrasonic endoscope 12, a foot switch operated by a foot, an apparatus for inputting information by a line of sight, or an apparatus capable of inputting information by voice can be used.

FIG. 5 illustrates a state where two measurement points (a first measurement point A and a second measurement point B) are specified for an ultrasound image G being displayed on the monitor 20 by the operation of the operator. In FIG. 5, a pointer P (A) indicating the first measurement point A and a pointer P (B) indicating the second measurement point B are displayed on the ultrasound image G in an overlapping manner. The ultrasound image G has a region T suspected of being a lesion.

As illustrated in FIG. 5, in a case where the first measurement point A and the second measurement point B are specified by the operator, the measurement unit 158B displays a straight line L1a connecting the first measurement point A and the second measurement point B on the ultrasound image G in an overlapping manner, further measures the length (distance between first measurement point A and second measurement point B) of the straight line L1a, and displays the measurement result on the ultrasound image G as a provisional measurement result (the result is “Distance 8.0 mm” in the example of FIG. 5) in an overlapping manner.

Operation for Generating Correction Support Information

In a case where two measurement points are specified as illustrated in FIG. 5, in the ultrasound image G, the correction support information generation unit 158A extends the straight line L1a from the first measurement point A to the side opposite to the second measurement point B, sets the first straight line L1 (a straight line consisting of a straight line L1a, a straight line L1b, and a straight line L1c) extending from the second measurement point B to the side opposite to the first measurement point A side, and extracts a range AR including the first straight line L1 (a rectangular range in the example of FIG. 5) as an analysis image. In FIG. 5, the straight line L1b, the straight line L1c, and the range AR1 are illustrated for explanation, and are not actually displayed on the monitor 20.

Next, the correction support information generation unit 158A performs smoothing to reduce noise of the analysis image. It is desirable that the correction support information generation unit 158A performs smoothing on the analysis image in a direction perpendicular to the first straight line L1. The correction support information generation unit 158A may perform smoothing using a two-dimensional filter (such as a bilateral filter) for storing image edges or another method. After smoothing, the correction support information generation unit 158A generates a brightness profile (first brightness profile) on the first straight line L1 in the analysis image.

FIG. 6 illustrates an example of a first brightness profile of a first straight line L illustrated in FIG. 5. A lateral axis of a graph illustrated in FIG. 6 indicates a position on the first straight line L1, and a vertical axis indicates a brightness value. In the graph illustrated in FIG. 6, the position of the first measurement point A is described as “A”, and the position of the second measurement point B is described as “B”.

The correction support information generation unit 158A detects a point (a first measurement candidate point Ax) on the first straight line L1 that is a correction candidate of the first measurement point A from a first range A1 on the basis of brightness change amount in the first range A1 including the first measurement point A in the first brightness profile illustrated in FIG. 6. In addition, the correction support information generation unit 158A detects a point (a second measurement candidate point Bx) on the first straight line L1 that is a correction candidate of the second measurement point B from a second range B1 on the basis of brightness change amount in the second range B1 including the second measurement point B in the first brightness profile.

The first range A1 is a range over the first measurement point A, and a center position of the range coincides with the position of the first measurement point A. The center position of the first range A1 does not need to coincide with the position of the first measurement point A.

The second range B1 is a range over the second measurement point B, and a center position of the range coincides with the position of the second measurement point B. The center position of the second range B1 does not need to coincide with the position of the second measurement point B.

Specific Example of Method for Detecting First Measurement Candidate Point Ax

The correction support information generation unit 158A calculates a brightness difference ΔY1 between each position of the first range A1 in the first brightness profile and an adjacent position in the direction from the first measurement point A toward the second measurement point B at each position, and in a case where there is a position where the brightness difference ΔY1 is equal to or more than a threshold value TH, determines the position as the first measurement candidate point Ax. In a case where there is a plurality of positions where the brightness difference ΔY1 is equal to or more than the threshold value TH, the correction support information generation unit 158A may determine a position at which the distance from the first measurement point A is minimum as the first measurement candidate point Ax.

In addition, in the case where there is the position where the brightness difference ΔY1 is equal to or more than the threshold value TH, the correction support information generation unit 158A calculates the reliability of the position on the basis of the distance from the first measurement point A of the position and the brightness difference ΔY1 between the position and the adjacent position. The reliability is determined to be higher as the brightness difference ΔY1 is larger and the distance from the first measurement point A is smaller. In the case where there are the plurality of positions where the brightness difference ΔY1 is equal to or more than the threshold value TH, the correction support information generation unit 158A may determine a position where the reliability is maximum, as the first measurement candidate point Ax, instead of the distance from the first measurement point A.

The correction support information generation unit 158A calculates an average brightness between the first measurement point A and the second measurement point B in the first brightness profile, or an average brightness near an intermediate position between the first measurement point A and the second measurement point B. Then, in the case where there is the position where the brightness difference ΔY1 is equal to or more than the threshold value TH, it is preferable that the correction support information generation unit 158A does not determine the brightness value of the adjacent position on the side opposite (left side in the example of FIG. 6) to the second measurement point B side than the position as the first measurement candidate point Ax, and in a case where the brightness is lower than the average brightness, it is preferable that the correction support information generation unit 158A does not determine the position as the first measurement candidate point Ax.

The correction support information generation unit 158A detects the first measurement candidate point Ax for the first range A1 as described above. In the case where the first measurement candidate point Ax is detected, the correction support information generation unit 158A stores position information (coordinates) of the first measurement candidate point Ax and the reliability calculated for the first measurement candidate point Ax.

Specific Example of Method for Detecting Second Measurement Candidate Point Bx

The correction support information generation unit 158A calculates a brightness difference ΔY2 between each position of the second range B1 in the first brightness profile and an adjacent position in the direction from the first measurement point A toward the second measurement point B at each position, and in a case where there is a position where the brightness difference ΔY2 is equal to or more than a threshold value TH, determines the position as the second measurement candidate point Bx. In a case where there is a plurality of positions where the brightness difference ΔY2 is equal to or more than the threshold value TH, the correction support information generation unit 158A may determine a position at which the distance from the second measurement point B is minimum as the second measurement candidate point Bx.

In addition, in the case where there is the position where the brightness difference ΔY2 is equal to or more than the threshold value TH, the correction support information generation unit 158A calculates the reliability of the position on the basis of the distance from the second measurement point B of the position and the brightness difference ΔY2 between the position and the adjacent position. The reliability is determined to be higher as the brightness difference ΔY2 is larger and the distance from the second measurement point B is smaller. In the case where there are the plurality of positions where the brightness difference ΔY2 is equal to or more than the threshold value TH, the correction support information generation unit 158A may determine a position where the reliability is maximum, as the second measurement candidate point Bx, instead of the distance from the second measurement point B.

The correction support information generation unit 158A calculates the above-described average brightness. Then, in the case where there is the position where the brightness difference ΔY2 is equal to or more than the threshold value TH, it is preferable that the correction support information generation unit 158A does not determine the brightness value of the adjacent position on the side opposite (right side in the example of FIG. 6) to the first measurement point A side than the position as the second measurement candidate point Bx, and in a case where the brightness is lower than the average brightness, it is preferable that the correction support information generation unit 158A does not determine the position as the second measurement candidate point Bx.

The correction support information generation unit 158A detects the second measurement candidate point Bx for the second range B1 as described above. In the case where the second measurement candidate point Bx is detected, the correction support information generation unit 158A stores position information (coordinates) of the second measurement candidate point Bx and the reliability calculated for the second measurement candidate point Bx.

In the example of FIG. 6, since a brightness variation in the first range A1 is small, the first measurement candidate point Ax is not detected from the first range A1. On the other hand, since a brightness of the second range B1 sharply increases at a position Bx, the position Bx is detected as the second measurement candidate point Bx. The position information of the first measurement candidate point Ax and the position information of the second measurement candidate point Bx detected by the correction support information generation unit 158A each constitute the first correction support information.

In a case where at least one of the first measurement candidate point Ax or the second measurement candidate point Bx is detected by the correction support information generation unit 158A, the measurement unit 158B displays the measurement candidate point on the monitor 20 on the basis of position information of the detected measurement candidate point. In addition, the measurement unit 158B measures a distance between one of the first measurement point A or the first measurement candidate point Ax and one of the second measurement point B or the second measurement candidate point Bx, and superimposes and displays the measurement result and the reliability of the first measurement candidate point Ax or the second measurement candidate point Bx on the ultrasound image G of the monitor 20 in an overlapping manner.

In the example of FIG. 6, only the second measurement candidate point Bx is detected. Therefore, the monitor 20 transitions from the state of FIG. 5 to the state of FIG. 7 under the control of the measurement unit 158B. In FIG. 7, a pointer P (Bx) indicating the second measurement candidate point Bx detected by the correction support information generation unit 158A is displayed on the ultrasound image G in an overlapping manner. In addition, in addition to the measurement result (8.0 mm) of the distance between the first measurement point A and the second measurement point B, the measurement result of the distance between the second measurement candidate point Bx and the first measurement point A and character of “9.5 mm: reliability 80%” indicating the reliability of the second measurement candidate point Bx are displayed on the ultrasound image G in an overlapping manner.

In a case where both the first measurement candidate point Ax and the second measurement candidate point Bx is detected by the correction support information generation unit 158A, the measurement unit 158B displays the measurement candidate point on the monitor 20 on the basis of position information of the detected measurement candidate point. In addition, the measurement unit 158B measures the distance between the second measurement candidate point Bx and the first measurement candidate point Ax, and displays the measurement result, the reliability of the second measurement candidate point Bx, and the reliability of the first measurement candidate point Ax on the ultrasound image G of the monitor 20 in an overlapping manner. FIG. 8 illustrates an example of a screen displayed on the monitor 20 in a case where both the first measurement candidate point Ax and the second measurement candidate point Bx are detected by the correction support information generation unit 158A.

On the screen illustrated in FIG. 8, a pointer P (Ax) indicating the first measurement candidate point Ax and the pointer P (Bx) indicating the second measurement candidate point Bx detected by the correction support information generation unit 158A are displayed on the ultrasound image G in an overlapping manner. In addition, in addition to the measurement result (8.0 mm) of the distance between the first measurement point A and the second measurement point B, a measurement result of a distance between the first measurement candidate point Ax and the second measurement candidate point Bx and character of “9.8 mm: reliability Ax 90%, reliability Bx 80%” indicating the reliability of the first measurement candidate point Ax and the second measurement candidate point Bx are displayed on the ultrasound image G in an overlapping manner.

In a case where the reliability of the first measurement candidate point Ax and the second measurement candidate point Bx is displayed as illustrated in FIG. 8, the average value of the reliability of the first measurement candidate point Ax and the second measurement candidate point Bx may be displayed.

In a case where the screen as illustrated in FIG. 7 or 8 is displayed, the operator performs an operation of determining two measurement points on the ultrasound image G. In a case where the set button 104 is pressed by the operator while the pointer P (A), the pointer P (B), and the pointer P (Bx) are displayed as illustrated in FIG. 7, the measurement unit 158B sets the first measurement point A as a first final measurement point, and sets the second measurement candidate point Bx as a second final measurement point. In this case, the second measurement candidate point Bx is a second corrected measurement point corrected from the second measurement point B on the basis of the first correction support information. On the other hand, in a case where the delete button 105 is pressed by the operator in this state, the measurement unit 158B sets the first measurement point A as a first final measurement point, and sets the second measurement point B as a second final measurement point. The operation for inputting the instruction to determine the measurement point described here may be performed using the above-described input apparatus other than the console 100.

In addition, in a case where the set button 104 is pressed by the operator while the pointer P (A), the pointer P (B), the pointer P (Ax), and the pointer P (Bx) are displayed as illustrated in FIG. 8, the measurement unit 158B sets the first measurement candidate point Ax as a first final measurement point, and sets the second measurement candidate point Bx as a second final measurement point. In this case, the first measurement candidate point Ax is a first corrected measurement point corrected from the first measurement point A on the basis of the first correction support information, and the second measurement candidate point Bx is a second corrected measurement point corrected from the second measurement point B on the basis of the first correction support information. On the other hand, in a case where the delete button 105 is pressed by the operator in this state, the measurement unit 158B sets the first measurement point A as a first final measurement point, and sets the second measurement point B as a second final measurement point. As described above, the operation for inputting the instruction to determine the measurement point described here may be performed using the above-described input apparatus other than the console 100.

In the state where the pointers P (A), P (B), P (Ax), and P (Bx) are displayed as illustrated in FIG. 8, in a case where either the first measurement point A or the first measurement candidate point Ax is selected on the touch pad 102 and the set button 104 is pressed, the measurement unit 158B may set one of them as the first final measurement point, and in a case where either the second measurement point B or the second measurement candidate point Bx is selected on the touch pad 102 and the set button 104 is pressed, the measurement unit 158B may set one of them as the second final measurement point.

In the case of setting the first final measurement point and the second final measurement point, the measurement unit 158B displays the pointer P indicating these and the straight line L1a connecting them on the ultrasound image G in an overlapping manner, and further displays a measurement result of a distance between them on the ultrasound image G in an overlapping manner. FIG. 9 illustrates an example of a screen displayed on the monitor 20 in a case where a set button 104 is pressed on the screen illustrated in FIG. 7. In the example of FIG. 9, the distance between the first measurement point A and the second measurement candidate point Bx is displayed as the measurement result.

Effect of Distance Measurement Mode of Ultrasonic Endoscope Apparatus 10

According to the ultrasonic endoscope apparatus 10, information indicating at least one position of a first measurement candidate point that is a correction candidate for a first measurement point or a second measurement candidate point that is a correction candidate for a second measurement point is generated and displayed on the monitor 20 as the first correction support information for supporting correction of the position of at least one of the first measurement point or the second measurement point on the ultrasound image specified by the operator. For example, even in a case where the operator specifies the first measurement point and the second measurement point with a margin outside a range suspected of a lesion site, a measurement candidate point is displayed near an edge position in the range. For this reason, even in a case of using an interface such as the touch pad 102 that requires time for detailed position specification, it is possible to accurately specify the desired range and measure the size of the range without spending time. Accordingly, the measurement range can be accurately specified within a limited time during the test, and the test efficiency and the reliability of the measurement result can be improved.

Further, according to the ultrasonic endoscope apparatus 10, the reliability of the measurement candidate point is displayed on the monitor 20 together with the position of the measurement candidate point. It is possible to assist in determining whether or not to determine the measurement candidate point as the final measurement point, and to prevent a range largely deviating from the operator's intention from being determined as the measurement range by displaying the reliability.

In addition, according to the ultrasonic endoscope apparatus 10, as illustrated in FIG. 6, the first range A1 used for detecting the first measurement candidate point is a range including the first measurement point A, and the second range B1 used for detecting the second measurement candidate point is a range including the second measurement point B. In this way, the detection of the first measurement candidate point or the second measurement candidate point is limited to around the first measurement point or the second measurement point specified by the operator. For this reason, even in a case where a region similar to the region T exists at a distant position next to the direction in which the first straight line L1 extends for the region T illustrated in FIG. 5, it is possible to prevent the measurement candidate point from being set at an edge part of the region, and to improve a measurement accuracy of the region T.

Further, according to the ultrasonic endoscope apparatus 10, the first range A1 is a range over the first measurement point A, and is preferably a bilaterally symmetrical range. In addition, the second range B1 is a range over the second measurement point B, and is preferably a bilaterally symmetrical range. Therefore, for example, compared to a case where the first range A1 is inside the first measurement point A and the second range B1 is inside the second measurement point B, the possibility that the measurement candidate point is detected can be increased, and the correction of the measurement point can be more strongly supported.

The size of each of the first range A1 and the second range B1 illustrated in FIG. 6 is not a fixed value but may be changeable. For example, the size of each of the first range A1 and the second range B1 may be randomly changed by the operator according to the operation of the console 100.

In addition, the size of each of the first range A1 and the second range B1 may be automatically changed according to an operating condition of the ultrasonic endoscope apparatus 10 (setting state of the ultrasound image generation mode), an observation target site (pancreas, gall bladder, or the like) by the ultrasonic endoscope apparatus 10, an attribute (medical history, age, and the like) of an observation target person by the ultrasonic endoscope apparatus 10, or a combination thereof. As described above, it is possible to detect an optimum measurement candidate point according to the situation, and to improve the reliability of the measurement result by making the size of each of the first range A1 and the second range B1 variable.

In the above description, the correction support information generation unit 158A detects the measurement candidate point by analyzing the first brightness profile. As a modification example, a learned model which sets at least the first measurement point, the second measurement point, and the first brightness profile as an input and outputs at least one of the first measurement candidate point or the second measurement candidate point may be prepared, and the measurement candidate point may be detected using the learned model.

For example, the learned model is added to the inside of the ultrasonic processor apparatus 14, and the correction support information generation unit 158A inputs the first measurement point, the second measurement point, and the first brightness profile to the learned model. In addition, in a case where one or both of the first measurement candidate point Ax and the second measurement candidate point Bx are output from the learned model, the correction support information generation unit 158A may acquire information on them and use the information on them as first correction support information.

The learned model described above may further learn not only the first measurement point, the second measurement point, and the first brightness profile but also an operating condition, an observation target site, and an attribute of an observation target person of the ultrasonic endoscope apparatus 10 at the time of acquiring the ultrasound image G in which the first measurement point and the second measurement point are specified. In addition, the learned model may be provided in the ultrasonic endoscope apparatus 10 and an external apparatus connected via a network.

First Modification Example of Operation in Distance Measurement Mode

In the above description, the correction support information generation unit 158A generates the position information of the first measurement candidate point Ax and the position information of the second measurement candidate point Bx as the first correction support information, and outputs them to the measurement unit 158B. The measurement unit 158B displays the position of the first measurement candidate point Ax and the position of the second measurement candidate point Bx on the ultrasound image G in an overlapping manner.

In the first modification example, the correction support information generation unit 158A generates, as first correction support information, a first brightness profile and information indicating a position of each of the first measurement point A, the second measurement point B, the first measurement candidate point Ax, and the second measurement candidate point Bx in the first brightness profile, and outputs them to the measurement unit 158B. Then, with the first brightness profile, the measurement unit 158B displays the position of the first measurement point A, the second measurement point B, the first measurement candidate point Ax, and the second measurement candidate point Bx on the first brightness profile on the ultrasound image G in an overlapping manner.

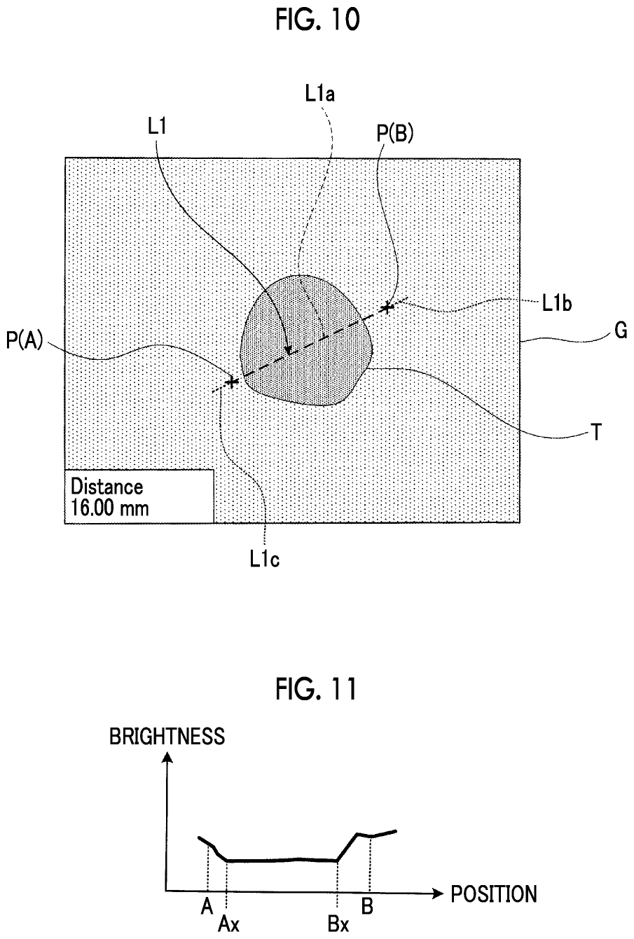

FIG. 10 is a schematic diagram illustrating another example of the screen displayed on the monitor 20 in the distance measurement mode. FIG. 10 illustrates a state where two measurement points (a first measurement point A and a second measurement point B) are specified for an ultrasound image G being displayed on the monitor 20 by the operation of the operator. In FIG. 10, a pointer P (A) indicating the first measurement point A and a pointer P (B) indicating the second measurement point B are displayed on the ultrasound image G in an overlapping manner.

As illustrated in FIG. 10, in a case where the first measurement point A and the second measurement point B are specified by the operator, the measurement unit 158B displays a straight line L1a connecting the first measurement point A and the second measurement point B on the ultrasound image G in an overlapping manner, further measures the length (distance between first measurement point A and second measurement point B) of the straight line L1a, and displays the measurement result on the ultrasound image G as a provisional measurement result (the result is “Distance 16.0 mm” in the example of FIG. 10) in an overlapping manner.

In a case where two measurement points are specified as illustrated in FIG. 10, in the ultrasound image G, the correction support information generation unit 158A extends the straight line L1a from the first measurement point A to the side opposite to the second measurement point B, sets the first straight line L1 (a straight line consisting of a straight line L1a, a straight line L1b, and a straight line L1c) extending from the second measurement point B to the side opposite to the first measurement point A side, and extracts a predetermined range including the first straight line L1 as an analysis image.

Then, by the same method as described above, the correction support information generation unit 158A generates the first brightness profile of the first straight line L1 using the analysis image, and detects the first measurement candidate point Ax and the second measurement candidate point Bx on the basis of the first brightness profile, the first measurement point A, and the second measurement point B. In addition, the correction support information generation unit 158A outputs information of the first brightness profile and information indicating the position of each of the first measurement point A, the second measurement point B, the first measurement candidate point Ax, and the second measurement candidate point Bx in the first brightness profile to the measurement unit 158B as the first correction support information.

FIG. 11 is a diagram illustrating an example of a first brightness profile of a first straight line L1 illustrated in FIG. 10.

In FIG. 11, an example in which the first measurement candidate point Ax is detected at a position closer to the second measurement point B than the first measurement point A, and the second measurement candidate point Bx is detected at a position closer to the first measurement point A than the second measurement point B is illustrated.

In a case of receiving the first correction support information from the correction support information generation unit 158A, the measurement unit 158B displays the first correction support information on the ultrasound image G of the monitor 20 in an overlapping manner.

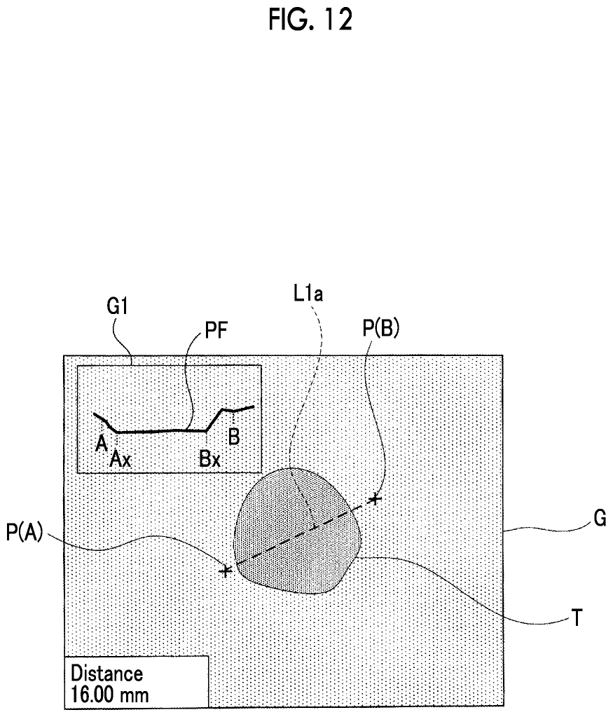

FIG. 12 is a schematic diagram illustrating an example of a screen displayed on the monitor 20 as a result of analyzing the first brightness profile illustrated in FIG. 11. As illustrated in FIG. 12, compared to the screen illustrated in FIG. 10, a subsidiary screen G1 is additionally displayed on the monitor 20. The subsidiary screen G1 includes a graph PF indicating the first brightness profile illustrated in FIG. 11, and positions of the first measurement point A, the second measurement point B, the first measurement candidate point Ax, and the second measurement candidate point Bx in the graph PF are illustrated by broken lines and characters.

In a case where the position information of the first measurement candidate point Ax is not included in the first correction support information, the information of the position of the first measurement candidate point Ax is not displayed on the subsidiary screen G1 in FIG. 12. Similarly, in a case where the position information of the second measurement candidate point Bx is not included in the first correction support information, the information of the position of the second measurement candidate point Bx is not displayed on the subsidiary screen G1 in FIG. 12.

In a case where the screen as illustrated in FIG. 12 is displayed, the operator performs an operation of determining two measurement points on the ultrasound image G. Specifically, in a case where the set button 104 is pressed by the operator while the screen illustrated in FIG. 12 is displayed, the measurement unit 158B sets the first measurement candidate point Ax as a first final measurement point, and sets the second measurement candidate point Bx as a second final measurement point. In this case, the first measurement candidate point Ax is the first corrected measurement point corrected from the first measurement point A on the basis of the first correction support information, and the second measurement candidate point Bx is the second corrected measurement point corrected from the second measurement point B on the basis of the first correction support information (in other words, the subsidiary screen G2). On the other hand, in a case where the delete button 105 is pressed by the operator in this state, the measurement unit 158B sets the first measurement point A as a first final measurement point, and sets the second measurement point B as a second final measurement point.

In a case where the set button 104 is pressed by the operator while the position information of the second measurement candidate point Bx is not included in the subsidiary screen G1 illustrated in FIG. 12, the measurement unit 158B sets the first measurement candidate point Ax as the first final measurement point, and sets the second measurement point B as the second final measurement point. In this case, the first measurement candidate point Ax is the first corrected measurement point corrected from the first measurement point A on the basis of the first correction support information.

In addition, in a case where the set button 104 is pressed by the operator while the position information of the first measurement candidate point Ax is not included in the subsidiary screen G1 illustrated in FIG. 12, the measurement unit 158B sets the first measurement point A as the first final measurement point, and sets the second measurement candidate point Bx as the second final measurement point. In this case, the second measurement candidate point Bx is a second corrected measurement point corrected from the second measurement point B on the basis of the first correction support information.

In addition, in a case where the set button 104 is pressed by the operator while the position information of the first measurement candidate point Ax and the second measurement candidate point Bx is not included in the subsidiary screen G1 illustrated in FIG. 12, the measurement unit 158B sets the first measurement point A as a first final measurement point, and sets the second measurement point B as a second final measurement point.

In this way, in the case of setting the first final measurement point and the second final measurement point, the measurement unit 158B displays the pointer P indicating the position of them and the straight line L1a connecting them on the ultrasound image G in an overlapping manner, and further displays the measurement result of the distance between them on the ultrasound image G in an overlapping manner.

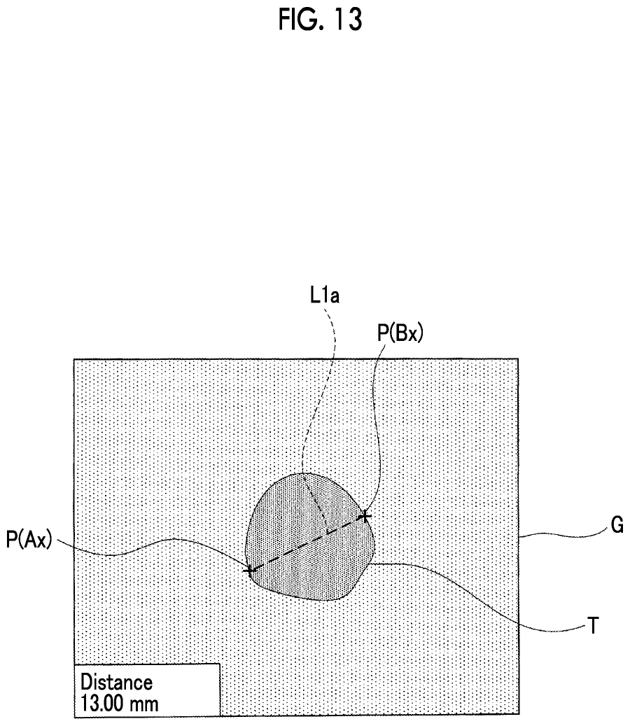

FIG. 13 illustrates an example of a screen displayed on the monitor 20 in a case where a set button 104 is pressed on the screen illustrated in FIG. 12. In the example of FIG. 13, the pointer P (Ax) indicating first measurement candidate point Ax and the pointer P (Bx) indicating second measurement candidate point Bx, the straight line L1a connecting the first measurement candidate point Ax and the second measurement candidate point Bx, and a measurement result (Distance 13.00 mm) of a distance between first measurement candidate point Ax and second measurement candidate point Bx are displayed on the ultrasound image G in an overlapping manner.

According to the operation of the above first modification example, it is possible to intuitively grasp where two measurement points specified by the operator and the measurement candidate point detected by apparatus are located on the first brightness profile by the subsidiary screen G1 illustrated in FIG. 12. Therefore, it is possible to easily determine whether or not the measurement candidate point should be set as the final measurement point. As a result, it is possible to improve test efficiency.

Second Modification Example of Operation in Distance Measurement Mode

In the second modification example, the correction support information generation unit 158A generates, as first correction support information, a first brightness profile and information indicating a position of each of the first measurement point A and the second measurement point B in the first brightness profile, and outputs them to the measurement unit 158B. Then, with the first brightness profile, the measurement unit 158B displays the position of the first measurement point A and the second measurement point B on the first brightness profile on the ultrasound image G in an overlapping manner on the basis of the first correction support information. Further, in a case where a position other than the first measurement point A and the second measurement point B on the first brightness profile displayed on the monitor 20 is specified by operating the console 100, the position is set as a first correction candidate point Axx of the first measurement point A or a second correction candidate point Bxx of the second measurement point B.

As illustrated in FIG. 10, the operation of the second modification example will be described by taking a case where the first measurement point A and the second measurement point B are specified by an operator as an example. As illustrated in FIG. 10, in a case where the first measurement point A and the second measurement point B are specified by the operator, the measurement unit 158B displays the straight line L1a connecting the first measurement point A and the second measurement point B on the ultrasound image G in an overlapping manner, further measures the length (distance between first measurement point A and second measurement point B) of the straight line L1a, and displays the measurement result on the ultrasound image G as the provisional measurement result (the result is “Distance 16.0 mm” in the example of FIG. 10) in an overlapping manner.

The correction support information generation unit 158A sets the first straight line L1 (a straight line consisting of the straight line L1a, the straight line L1b, and the straight line L1c) in the ultrasound image G, and extracts a predetermined range including the first straight line L1 as an analysis image. Then, the correction support information generation unit 158A generates the first brightness profile of the first straight line L1 using the analysis image in the same method as described above.

The correction support information generation unit 158A outputs information of the first brightness profile and information indicating the position of each of the first measurement point A and the second measurement point B in the first brightness profile to the measurement unit 158B as the first correction support information. The first brightness profile generated here is the same as that illustrated in FIG. 11.

In a case of receiving the first correction support information from the correction support information generation unit 158A, the measurement unit 158B displays the first correction support information on the ultrasound image G of the monitor 20 in an overlapping manner.

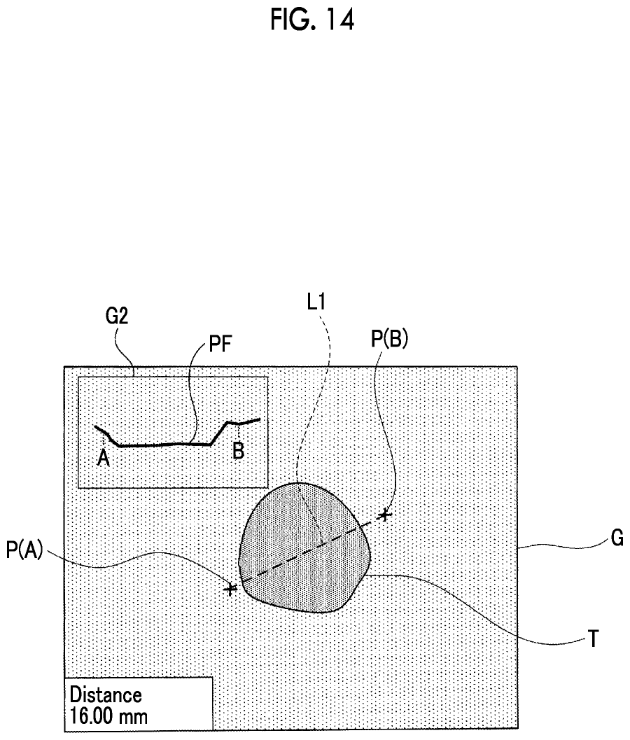

FIG. 14 illustrates a state where the first brightness profile illustrated in FIG. 11 and information indicating a position of the first measurement point A and the second measurement point B in the first brightness profile are additionally displayed on the monitor 20 with respect to the screen of FIG. 10. As illustrated in FIG. 14, compared to the screen illustrated in FIG. 10, a subsidiary screen G2 is additionally displayed on the monitor 20. The subsidiary screen G2 includes a graph PF indicating the first brightness profile illustrated in FIG. 11, and positions of each of the first measurement point A and the second measurement point B in the graph PF are illustrated by broken lines and characters.

In a case where the screen illustrated in FIG. 14 is displayed, an operator performs an operation of specified two measurement points on the graph PF in the subsidiary screen G2. Specifically, while the subsidiary screen G2 is displayed, the operator operates the touch pad 102 to select a random point (for example, a point between the first measurement point A and the second measurement point B and closer to the first measurement point A) on the graph PF with the pointer (not illustrated), and in this state, in a case where the set button 104 is pressed, the measurement unit 158B sets the selected point as a first correction candidate point Axx. In addition, the operator operates the touch pad 102 to select a random point (for example, a point between the first measurement point A and the second measurement point B and closer to the second measurement point B) on the graph PF using a pointer (not illustrated), and in this state, in a case where the set button 104 is pressed, the measurement unit 158B sets the selected point as a second correction candidate point Bxx.

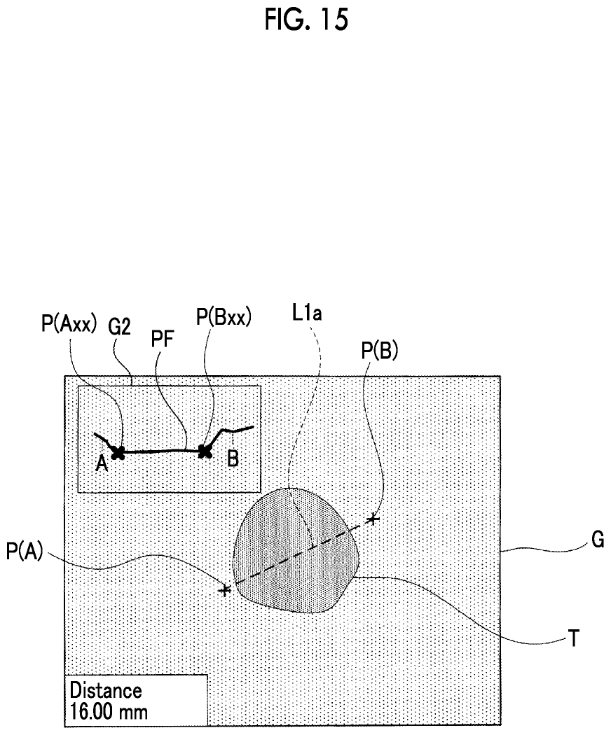

FIG. 15 illustrates a state where two points on a graph PF in a subsidiary screen G2 are selected on the screen illustrated in FIG. 14 and a set button 104 is pressed. As illustrated in FIG. 15, compared to the screen illustrated in FIG. 14, an X-shaped pointer P (Axx) indicating a position of the first correction candidate point Axx and an X-shaped pointer P (Bxx) indicating a position of the second correction candidate point Bxx are added to the subsidiary screen G2.

In a case where the set button 104 is pressed by the operator in the state illustrated in FIG. 15 is displayed, the measurement unit 158B sets the first correction candidate point Axx as a first final measurement point, and sets the second correction candidate point Bxx as a second final measurement point. In this case, the first correction candidate point Axx is the first corrected measurement point corrected from the first measurement point A on the basis of the first correction support information (in other words, the subsidiary screen G2). In this case, the second correction candidate point Bxx is the second corrected measurement point corrected from the second measurement point B on the basis of the first correction support information (in other words, the subsidiary screen G2).

In a case where the first final measurement point and the second final measurement point are set in this way, the measurement unit 158B deletes the subsidiary screen G2 from the ultrasound image G of the monitor 20, and further displays a pointer P (Ax) indicating the first final measurement point, a pointer P (Bx) indicating the second final measurement point, a straight line L1a connecting the first final measurement point and the second final measurement point, and a measurement result of the distance between the first and second final measurement points on the ultrasound image G in an overlapping manner, and switches the screen of the monitor 20 to, for example, the screen illustrated in FIG. 13.

In a case where the delete button 105 is pressed by the operator in the state illustrated in FIG. 15, the measurement unit 158B sets the first measurement point A as the first final measurement point, and sets the second measurement point B as the second final measurement point. In addition, in a case where the set button 104 is pressed by the operator without specifying a point on the graph PF in the state illustrated in FIG. 14, the measurement unit 158B sets the first measurement point A as the first final measurement point, and sets the second measurement point B as the second final measurement point.

In addition, in a case where the state shifts from the state illustrated in FIG. 14 to the state in which only the first correction candidate point Axx illustrated in FIG. 15 is set, and the set button 104 is pressed in the state, the measurement unit 158B sets the first correction candidate point Axx as the first final measurement point, and sets the second measurement point B as the second final measurement point. In this case, the first correction candidate point Axx is the first corrected measurement point corrected from the first measurement point A on the basis of the first correction support information.

In addition, in a case where the state shifts from the state illustrated in FIG. 14 to the state in which only the second correction candidate point Bxx illustrated in FIG. 15 is set, and the set button 104 is pressed in the state, the measurement unit 158B sets the first measurement point A as the first final measurement point, and sets the second correction candidate point Bxx as the second final measurement point. In this case, the second correction candidate point Bxx is the second corrected measurement point corrected from the second measurement point B on the basis of the first correction support information.

According to the operation of the above second modification example, it is possible to grasp a relationship between two measurement points specified by the operator and the first brightness profile by the subsidiary screen G2 illustrated in FIG. 14. Then, a correction candidate point can be manually selected on the subsidiary screen G2. According to the modification example, instead of displaying a measurement candidate point automatically detected by the apparatus, the operator can select a random point from the brightness profile and set it as the correction candidate point. Therefore, a flexible measurement range can be set in accordance with the operator's intention, and measurement accuracy can be improved. In addition, since the brightness profile and the two points selected by the operator are displayed together, it is possible to intuitively grasp a relationship between a boundary position of a region T and a position of the selected measurement point, and it becomes easy to specify a correction candidate point closer to the boundary position. As a result, an accurate measurement range can be quickly set, and test efficiency can be improved.

Operation in Area Measurement Mode

In the area measurement mode, the console 100 can be operated on the ultrasound image G displayed on the monitor 20 to specify an elliptical range. Specifically, first, an operator operates the touch pad 102 as described above to specify the first measurement point A and the second measurement point B.

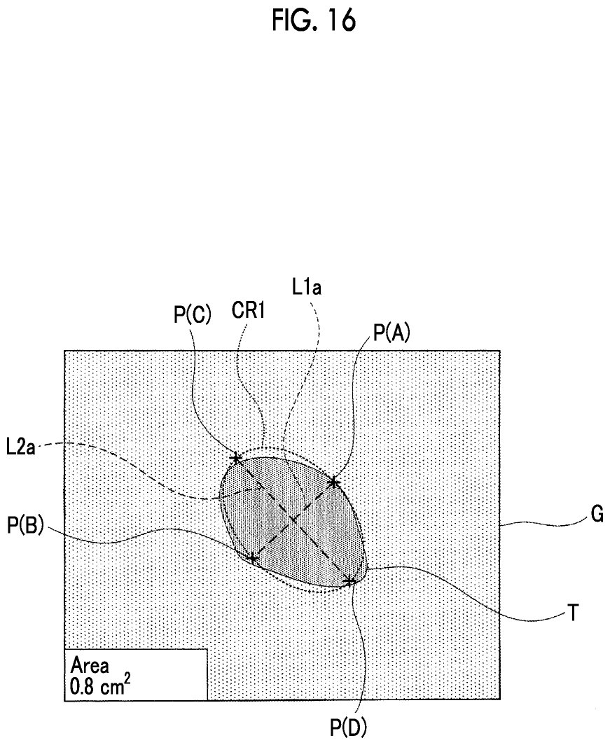

In a case of being specified, as illustrated in FIG. 16, the measurement unit 158B displays a straight line L1a connecting the first measurement point A and the second measurement point B, and a straight line L2a having a predetermined length that is orthogonal to the straight line L1a and passes through a midpoint of the straight line L1a on the ultrasound image G in an overlapping manner. Then, at one end of the straight line L2a, a pointer P (C) indicating a temporary third measurement point C is displayed, and at the other end of the straight line L2a, a pointer P (D) indicating a temporary fourth measurement point D is displayed. In addition, the measurement unit 158B also displays an ellipse CR1 having the straight line L1a as a short axis and the straight line L2a as a long axis. Further, the measurement unit 158B measures an area of the ellipse CR1, and displays the result (in the example of FIG. 16, “Area 0.8 cm²”).

In the state illustrated in FIG. 16, in a case where the operator moves, for example, a finger on the touch pad 102 in a random direction, the measurement unit 158B increases the length of the straight line L2a, and updates the display range and the area measurement result of the ellipse CR1 accordingly. In a case where the operator moves, for example, a finger on the touch pad 102 in a direction opposite to the above random direction, the measurement unit 158B shortens the length of the straight line L2a, and updates the display range and the area measurement result of the ellipse CR1 accordingly. Four points (first measurement point A, second measurement point B, third measurement point C, fourth measurement point D) on the ultrasound image G can be specified by operating the touch pad 102 in this way. Specifying the four measurement points described here may be performed using the above-described input apparatus other than the console 100.

In the state where the four points are specified as illustrated in FIG. 16, in a case where the set button 104 is pressed, the correction support information generation unit 158A generates first correction support information for supporting a correction of a position of at least one of a first measurement point A or a second measurement point B on a first straight line extending both ends of the straight line L1a by the same operation as the various operations described in the distance measurement mode.

In addition, the correction support information generation unit 158A generates second correction support information for supporting a correction of a position of at least one of the third measurement point C or the fourth measurement point D on a second straight line extending both ends of the straight line L2a outward by a predetermined amount. The content of the second correction support information and the method of generating the same are the same as those of the first correction support information, and thus description thereof will be omitted. A brightness profile of the second straight line constitutes a second brightness profile.

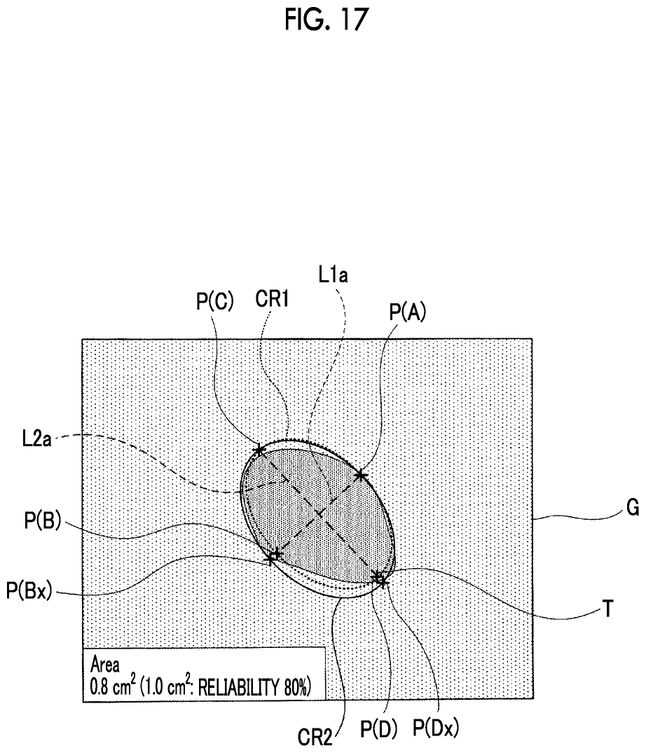

The measurement unit 158B displays the first correction support information and the second correction support information on the ultrasound image G of the monitor 20 in an overlapping manner. In FIG. 17, an example is illustrated in which a pointer P (Bx) indicating a position of a second measurement candidate point Bx which is a correction candidate of the second measurement point is displayed in an overlapping manner, as the first correction support information, and a pointer P (Dx) indicating a position of a fourth measurement candidate point Dx which is a correction candidate for the fourth measurement point is displayed in an overlapping manner, as the second correction support information.

The measurement unit 158B further causes the screen illustrated in FIG. 17 to display an ellipse CR2 passing through the first measurement point A, the second measurement candidate point Bx, the third measurement point C, and the fourth measurement candidate point Dx, measures an area of a range surrounded by the ellipse CR2, and causes the screen to display a measurement result (1.0 cm²). In addition, the measurement unit 158B calculates the reliability of the ellipse CR2, and displays the reliability (“reliability 80%” in the example of FIG. 17) on the ultrasound image G in an overlapping manner. For example, as the reliability of the ellipse CR2, in a case where only one measurement candidate point is detected, the reliability of the measurement candidate point is used, and in a case where a plurality of measurement candidate points are detected, an average value of the reliability of the plurality of measurement candidate points is used.

In addition, in a case where the set button 104 is pressed by the operator while the pointer P (A), the pointer P (Bx), the pointer P (C), and the pointer P (Dx) are displayed as illustrated in FIG. 17, the measurement unit 158B sets the first measurement point A as a first final measurement point, sets the second measurement candidate point Bx as a second final measurement point, sets the third measurement point C as a third final measurement point, and sets the fourth measurement candidate point Dx as a fourth final measurement point. The operation for inputting the instruction to determine the measurement point described here may be performed using the above-described input apparatus other than the console 100.

In this case, the second measurement candidate point Bx is a second corrected measurement point corrected from the second measurement point B on the basis of the first correction support information. Further, in this case, the fourth measurement candidate point Dx is a fourth corrected measurement point corrected from the fourth measurement point D on the basis of the second correction support information.

On the other hand, in a case where the delete button 105 is pressed by the operator in the state of FIG. 17, the measurement unit 158B sets the first measurement point A as the first final measurement point, and sets the second measurement point B as the second final measurement point, sets the third measurement point C as the third final measurement point, and sets the fourth measurement point D as the fourth final measurement point.

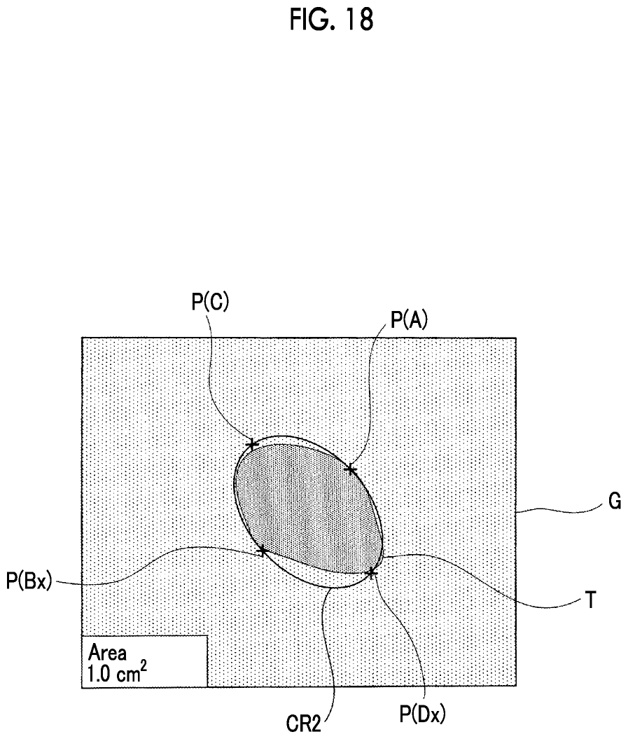

In a case where the set button 104 is pressed from the state illustrated in FIG. 17 to set the first final measurement point, the second final measurement point, the third final measurement point, and the fourth final measurement point, the measurement unit 158B displays, as illustrated in FIG. 18, a pointer P indicating these and an ellipse CR2 passing through them on the ultrasound image G in an overlapping manner, and Further, displays a measurement result of an area of a range surrounded by the ellipse CR2 on the ultrasound image G in an overlapping manner.

As described above, according to the ultrasonic endoscope apparatus 10, in the area measurement mode, similarly to the distance measurement mode, correction support information for correcting the measurement point specified by the operator is generated and displayed on the monitor 20. Therefore, specifying the elliptical range can be performed quickly and accurately. Therefore, it is possible to improve the test efficiency and the reliability of the measurement result.

Modification Example of Operation in Area Measurement Mode

In the modification example, after four measurement points are specified in the ultrasound image G, first, the first correction support information is generated and displayed, and then, the first final measurement point and the second final measurement point are set according to the operation of the operator. Thereafter, the second correction support information is generated and displayed, and then, the third final measurement point and the fourth final measurement point are set according to the operation of the operator.

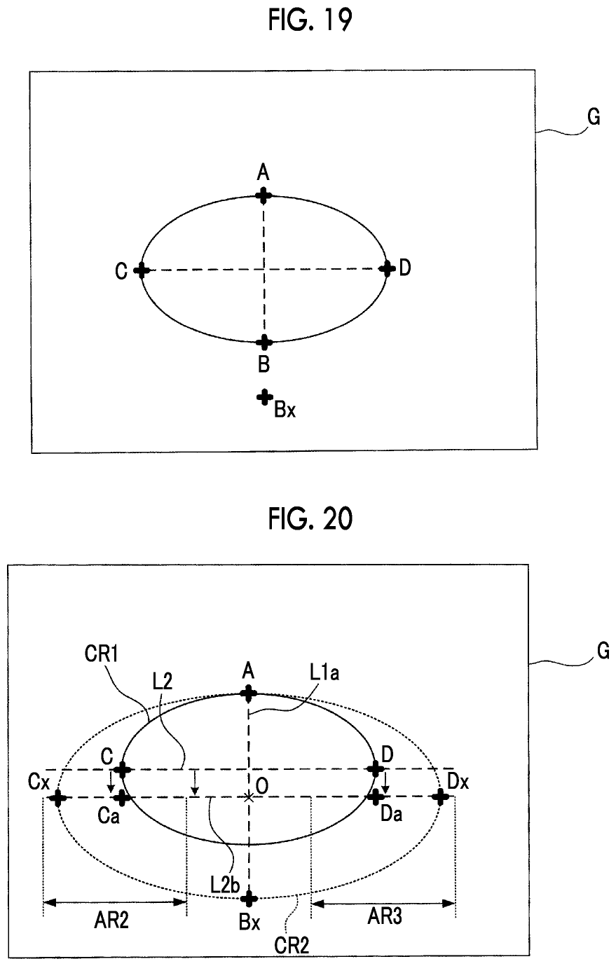

FIG. 19 illustrates an example in which a first measurement point A, a second measurement point B, a third measurement point C, and a fourth measurement point D are set on the ultrasound image G displayed on the monitor 20, and then, first, a second measurement candidate point Bx which is a correction candidate of the second measurement point B is displayed as the first correction support information.

In a case where the set button 104 is pressed in the state illustrated in FIG. 19, the measurement unit 158B sets the first measurement point A as the first final measurement point, and sets the second measurement candidate point Bx as the second final measurement point. Then, the measurement unit 158B displays the straight line L1a connecting the first measurement point A and the second measurement candidate point Bx on the ultrasound image G in an overlapping manner.

In this state, the correction support information generation unit 158A sets the above-described second straight line L2 passing through the third measurement point C and the fourth measurement point D. In a state (a state in FIG. 20 illustrates the state) where the second straight line L2 intersects other than a midpoint O of the straight line L1a which is a line segment connecting the first final measurement point and the second final measurement point, the correction support information generation unit 158A makes a state where the second straight line L2 intersects the midpoint O by moving the second straight line L2, the third measurement point C, and the fourth measurement point D in parallel to a direction along the straight line L1a. In FIG. 20, a straight line after the second straight line L2 is moved in parallel is defined as a second straight line L2b, a point after the third measurement point C is moved in parallel is defined as a third measurement point Ca, and a point after the fourth measurement point D is moved in parallel is defined as a fourth measurement point Da.

Then, the correction support information generation unit 158A detects a third measurement candidate point Cx and a fourth measurement candidate point Dx at equal distances from the midpoint O on the second straight line L2b by the above-described method. For example, the correction support information generation unit 158A calculates a brightness difference ΔYc between each position and an adjacent position in a range AR2 including the third measurement point Ca (see FIG. 20), and obtains a brightness difference ΔYd between each position and an adjacent position in a range AR3 including the fourth measurement point Da (see FIG. 20). The correction support information generation unit 158A calculates the reliability by subtracting a value obtained by multiplying a distance between the position and the third measurement point C or the fourth measurement point D by a random weighting coefficient from a sum of the brightness difference Yc and the brightness difference Yd described above at the corresponding position in the range AR2 and the range AR3 (the position at the same distance from one end of each range), with the sizes of the range AR2 and the range AR3 being the same. Then, the correction support information generation unit 158A detects, as the third measurement candidate point Cx and the fourth measurement candidate point Dx, a position having maximum reliability among positions where the reliability is equal to or more than a threshold value.

In a case where the third measurement candidate point Cx and the fourth measurement candidate point Dx at equal distances cannot be detected from the midpoint O on the second straight line L2b, the correction support information generation unit 158A does not cause the monitor 20 to display the third measurement candidate point Cx and the fourth measurement candidate point Dx.

In a case where the third measurement candidate point Cx and the fourth measurement candidate point Dx at equal distances can be detected from the midpoint O on the second straight line L2b, the correction support information generation unit 158A causes the monitor 20 to display pointers indicating the third measurement candidate point Cx and the fourth measurement candidate point Dx, as illustrated in FIG. 20.

In a case where the set button 104 is pressed in the state illustrated in FIG. 20, the measurement unit 158B sets the third measurement candidate point Cx as the third final measurement point, and sets the fourth measurement candidate point Dx as the fourth final measurement point. Then, the measurement unit 158B displays an ellipse CR2 on the ultrasound image G in an overlapping manner, in which the ellipse CR2 has a straight line connecting the third measurement candidate point Cx and the fourth measurement candidate point Dx as a long axis, and a straight line connecting the first measurement point A and the second measurement point B as a short axis. Further, the measurement unit 158B measures the area of the range surrounded by the ellipse CR2, and displays the measurement result on the ultrasound image G in an overlapping manner.

According to the operation of the modification example of the area measurement mode, an elliptical range having the straight line connecting the two measurement points specified by the operator as the short axis or the long axis can be presented to the operator as a candidate of a measurement target range. For this reason, the ellipse CR2 that is the candidate of the measurement target range as illustrated in FIG. 20 can be enlarged or reduced with respect to the range initially specified by the operator, starting from the first measurement point A, and a large deviation between the candidate of the measurement target range and the range specified by the operator can be prevented.

Each functional block of the measurement controller 158 in the above embodiment and the modification example may be configured to be provided in a processor included in the endoscopic processor apparatus 16, and may be configured to be provided in a processor included in an external apparatus such as an external server that can be connected to the ultrasonic endoscope apparatus 10.

As described above, the following items are disclosed in the embodiment of the present invention.

(1) A measurement apparatus comprising a correction support information generation unit that generates a first brightness profile on a first straight line passing through a first measurement point and a second measurement point specified for an ultrasound image generated by an ultrasound diagnostic apparatus and displayed on a display unit, and generates first correction support information for supporting correction of at least one position of the first measurement point or the second measurement point on the first straight line on the basis of the first brightness profile, and a measurement unit that displays the first correction support information on the display unit, and on the basis of instructions input in a state where the first correction support information is displayed on the display unit, determines one of the first measurement point or a first corrected measurement point corrected from the first measurement point based on the first correction support information as a first final measurement point, determines one of the second measurement point or a second corrected measurement point corrected from the second measurement point based on the first correction support information as a second final measurement point, and measures a size of a measurement range on the ultrasound image based on the first final measurement point and the second final measurement point.

(2) The measurement apparatus described in (1), in which the correction support information generation unit generates the first correction support information on the basis of the first measurement point, the second measurement point, and the first brightness profile.

(3) The measurement apparatus described in (2), in which the correction support information generation unit detects a first measurement candidate point that is a correction candidate of the first measurement point from a first range including the first measurement point in the first brightness profile on the basis of a brightness change amount of the first range, detects a second measurement candidate point that is a correction candidate of the second measurement point from a second range including the second measurement point in the first brightness profile on the basis of a brightness change amount of the second range, and outputs information indicating a position of one or both of the first measurement candidate point and the second measurement candidate point as the first correction support information.

(4) The measurement apparatus described in (3), in which the correction support information generation unit determines first reliability of the first measurement candidate point on the basis of a difference between a brightness value at a position of the first measurement candidate point and a brightness value at a position adjacent to the position of the first measurement candidate point and a distance between the first measurement candidate point and the first measurement point, determines second reliability of the second measurement candidate point on the basis of a difference between a brightness value at a position of the second measurement candidate point and a brightness value at a position adjacent to the position of the second measurement candidate point and a distance between the second measurement candidate point and the second measurement point, and outputs information indicating the first reliability and the second reliability as the first correction support information.

(5) The measurement apparatus described in (3) or (4), in which the first range is a range over the first measurement point, and the second range is a range over the second measurement point.

(6) The measurement apparatus described in any one of (3) to (5), in which a size of each of the first range and the second range is changeable.

(7) The measurement apparatus described in (6), in which the size of each of the first range and the second range is changed according to an operating condition of the ultrasound diagnostic apparatus, an observation target site by the ultrasound diagnostic apparatus, an attribute of an observation target person by the ultrasound diagnostic apparatus, or a combination thereof.

(8) The measurement apparatus described in (2), in which the correction support information generation unit detects a first measurement candidate point that is a correction candidate of the first measurement point from a first range including the first measurement point in the first brightness profile on the basis of a brightness change amount of the first range, detects a second measurement candidate point that is a correction candidate of the second measurement point from a second range including the second measurement point in the first brightness profile on the basis of a brightness change amount of the second range, and outputs the first brightness profile and information indicating positions of the first measurement candidate point, the second measurement candidate point, the first measurement point, and the second measurement point in the first brightness profile, as the first correction support information.

(9) The measurement apparatus described in (2), in which the correction support information generation unit acquires a first measurement candidate point that is a correction candidate of the first measurement point and a second measurement candidate point that is a correction candidate of the second measurement point from a learned model by inputting at least the first measurement point, the second measurement point, or the first brightness profile to a learned model, and outputs information including one or both positions of the first measurement candidate point and the second measurement candidate point as the first correction support information, the learned model outputs the first measurement candidate point and the second measurement candidate point by inputting at least the first measurement point, the second measurement point, or the first brightness profile.

(10) The measurement apparatus described in (2), in which the correction support information generation unit outputs the first brightness profile and information indicating positions of the first measurement point and the second measurement point in the first brightness profile as the first correction support information, and in which in a case where the point that is different from the first measurement point and the second measurement point and closer to the first measurement point than the second measurement point is specified for the first brightness profile displayed on the display unit, the measurement unit sets the point as the first corrected measurement point and in a case where a point that is different from the first measurement point and the second measurement point and closer to the second measurement point than the first measurement point is specified, sets the point as the second corrected measurement point.

(11) The measurement apparatus described in any one of (1) to (10), in which the correction support information generation unit generates a second brightness profile on a second straight line passing through a third measurement point and a fourth measurement point specified for the ultrasound image and is orthogonal to the first straight line, and generates second correction support information for supporting correction of at least one position of the third measurement point or the fourth measurement point on the second straight line on the basis of the second brightness profile, and in which the measurement unit displays the second correction support information on the display unit, and on the basis of instructions input in a state where the second correction support information is displayed on the display unit, determines one of the third measurement point or a third corrected measurement point corrected from the third measurement point based on the second correction support information as a third final measurement point, determines one of the fourth measurement point or a fourth corrected measurement point corrected from the fourth measurement point based on the second correction support information as a fourth final measurement point, and measures an area of an elliptical measurement range passing through the first final measurement point, the second final measurement point, the third final measurement point, and the fourth final measurement point in the ultrasound image.

(12) The measurement apparatus described in (11), in which the correction support information generation unit detects a third measurement candidate point that is a correction candidate of the third measurement point from a third range including the third measurement point in the second brightness profile on the basis of a brightness change amount of the third range, detects a fourth measurement candidate point that is a correction candidate of the fourth measurement point from a fourth range including the fourth measurement point in the second brightness profile on the basis of a brightness change amount of the fourth range, and generates information including one or both positions of the third measurement candidate point and the fourth measurement candidate point as the second correction support information, and further in which, in a state where the second straight line intersects points other than a midpoint of a line segment connecting the first final measurement point and the second final measurement point, the correction support information generation unit detects the third measurement candidate point and the fourth measurement candidate point at equal distances from the midpoint by moving the second straight line, the third measurement point, and the fourth measurement point in parallel along the line segment to have a state where the second straight line intersects the midpoint.

(13) The ultrasound diagnostic apparatus comprising the measurement apparatus described in any one of (1) to (12), and an image processing unit that generates an ultrasound image based on an output signal of an ultrasonic endoscope.

(14) A measurement method comprising a correction support information generation step of generating a first brightness profile on a first straight line passing through a first measurement point and a second measurement point specified for an ultrasound image generated by an ultrasound diagnostic apparatus and displayed on a display unit, and generating first correction support information for supporting correction of at least one position of the first measurement point or the second measurement point on the first straight line on the basis of the first brightness profile, and a measurement step of displaying the first correction support information on the display unit, and on the basis of instructions input in a state where the first correction support information is displayed on the display unit, determining one of the first measurement point or a first corrected measurement point corrected from the first measurement point based on the first correction support information as a first final measurement point, determining one of the second measurement point or a second corrected measurement point corrected from the second measurement point based on the first correction support information as a second final measurement point, and measuring a size of a measurement range on the ultrasound image based on the first final measurement point and the second final measurement point.

(15) A non-transitory computer readable recording medium storing a measurement program that causes a computer to perform a correction support information generation step of generating a first brightness profile on a first straight line passing through a first measurement point and a second measurement point specified for an ultrasound image generated by an ultrasound diagnostic apparatus and displayed on a display unit, and generating first correction support information for supporting correction of at least one position of the first measurement point or the second measurement point on the first straight line on the basis of the first brightness profile, and a measurement step of displaying the first correction support information on the display unit, and on the basis of instructions input in a state where the first correction support information is displayed on the display unit, determining one of the first measurement point or a first corrected measurement point corrected from the first measurement point based on the first correction support information as a first final measurement point, determining one of the second measurement point or a second corrected measurement point corrected from the second measurement point based on the first correction support information as a second final measurement point, and measuring a size of a measurement range on the ultrasound image based on the first final measurement point and the second final measurement point.

### EXPLANATION OF REFERENCES

10: ultrasonic endoscope apparatus
12: ultrasonic endoscope
14: ultrasonic processor apparatus
16: endoscopic processor apparatus
18: light source apparatus
20: monitor
21a: water supply tank
21b: suction pump
21c: air supply pump
22: insertion part
24: operation unit
26: universal cord
28a: air and water supply button
28b: suction button
30: treatment instrument insertion port
32a: ultrasonic connector
32b: endoscopic connector
32c: light source connector
36: ultrasonic observation part
37: balloon
38: endoscopic observation part
40: distal end part
42: bending part
43: flexible part
46: ultrasound transducer unit
100: console
101: touch panel
102: touch pad
103: measure button
104: set button
105: delete button
107: area button
108: distance button
140: multiplexer
142: receiving circuit
144: transmitting circuit
146: A/D converter
148: image processing unit
152: system controller
154: digital scan converter (DSC)
156: cine memory
158: measurement controller
158A: correction support information generation unit
158B: measurement unit
A: first measurement point
B: second measurement point
C: third measurement point
D: fourth measurement point
Ax: first measurement candidate point
Bx: second measurement candidate point
Cx: third measurement candidate point
Dx: fourth measurement candidate point
L1a, L1b, L1c, L2a: straight line
AR1, AR2, AR3: range
A1: first range
B1: second range
P (A), P (B), P (C), P (D): pointer
P (Ax), P (Bx), P (Axx), P (Bxx), P (Dx): pointer
T: region
G: ultrasound image
PF: graph
G1, G2: subsidiary screen
CR1, CR2: ellipse

What is claimed is:

1. A measurement apparatus comprising:
a correction support information generation unit that generates a first brightness profile on a first straight line passing through a first measurement point and a second measurement point specified for an ultrasound image generated by an ultrasound diagnostic apparatus and displayed on a display unit, and generates first correction support information for supporting correction of at least one position of the first measurement point or the second measurement point on the first straight line on the basis of the first brightness profile; and
a measurement unit that displays the first correction support information on the display unit, and on the basis of instructions input in a state where the first correction support information is displayed on the display unit, determines one of the first measurement point or a first corrected measurement point corrected from the first measurement point based on the first correction support information as a first final measurement point, determines one of the second measurement point or a second corrected measurement point corrected from the second measurement point based on the first correction support information as a second final measurement point, and measures a size of a measurement range on the ultrasound image based on the first final measurement point and the second final measurement point.

2. The measurement apparatus according to claim 1,
wherein the correction support information generation unit generates the first correction support information on the basis of the first measurement point, the second measurement point, and the first brightness profile.

3. The measurement apparatus according to claim 2,
wherein the correction support information generation unit detects a first measurement candidate point that is a correction candidate of the first measurement point from a first range including the first measurement point in the first brightness profile on the basis of a brightness change amount of the first range, detects a second measurement candidate point that is a correction candidate of the second measurement point from a second range including the second measurement point in the first brightness profile on the basis of a brightness change amount of the second range, and outputs information indicating a position of one or both of the first measurement candidate point and the second measurement candidate point as the first correction support information.

4. The measurement apparatus according to claim 3,
wherein the correction support information generation unit determines first reliability of the first measurement candidate point on the basis of a difference between a brightness value at a position of the first measurement candidate point and a brightness value at a position adjacent to the position of the first measurement candidate point and a distance between the first measurement candidate point and the first measurement point, determines second reliability of the second measurement candidate point on the basis of a difference between a brightness value at a position of the second measurement candidate point and a brightness value at a position adjacent to the position of the second measurement candidate point and a distance between the second measurement candidate point and the second measurement point, and outputs information indicating the first reliability and the second reliability as the first correction support information.

5. The measurement apparatus according to claim 3,
wherein the first range is a range over the first measurement point, and the second range is a range over the second measurement point.

6. The measurement apparatus according to claim 4,
wherein the first range is a range over the first measurement point, and the second range is a range over the second measurement point.

7. The measurement apparatus according to claim 3,
wherein a size of each of the first range and the second range is changeable.

8. The measurement apparatus according to claim 4,
wherein a size of each of the first range and the second range is changeable.

9. The measurement apparatus according to claim 5,
wherein a size of each of the first range and the second range is changeable.

10. The measurement apparatus according to claim 6,
wherein a size of each of the first range and the second range is changeable.

11. The measurement apparatus according to claim 7,
wherein the size of each of the first range and the second range is changed according to an operating condition of the ultrasound diagnostic apparatus, an observation target site by the ultrasound diagnostic apparatus, an attribute of an observation target person by the ultrasound diagnostic apparatus, or a combination thereof.

12. The measurement apparatus according to claim 8,
wherein the size of each of the first range and the second range is changed according to an operating condition of the ultrasound diagnostic apparatus, an observation target site by the ultrasound diagnostic apparatus, an attribute of an observation target person by the ultrasound diagnostic apparatus, or a combination thereof.

13. The measurement apparatus according to claim 2,
wherein the correction support information generation unit detects a first measurement candidate point that is a correction candidate of the first measurement point from a first range including the first measurement point in the first brightness profile on the basis of a brightness change amount of the first range, detects a second measurement candidate point that is a correction candidate of the second measurement point from a second range including the second measurement point in the first brightness profile on the basis of a brightness change amount of the second range, and outputs the first brightness profile and information indicating positions of the first measurement candidate point, the second measurement candidate point, the first measurement point, and the second measurement point in the first brightness profile, as the first correction support information.

14. The measurement apparatus according to claim 2,
wherein the correction support information generation unit acquires a first measurement candidate point that is a correction candidate of the first measurement point and a second measurement candidate point that is a correction candidate of the second measurement point from a learned model by inputting at least the first measurement point, the second measurement point, or the first brightness profile to a learned model, and outputs information including one or both positions of the first measurement candidate point and the second measurement candidate point as the first correction support information, the learned model outputs the first measurement candidate point and the second measurement candidate point by inputting at least the first measurement point, the second measurement point, or the first brightness profile.

15. The measurement apparatus according to claim 2,
wherein the correction support information generation unit outputs the first brightness profile and information indicating positions of the first measurement point and the second measurement point in the first brightness profile as the first correction support information, and
wherein in a case where a point that is different from the first measurement point and the second measurement point and closer to the first measurement point than the second measurement point is specified for the first brightness profile displayed on the display unit, the measurement unit sets the point as the first corrected measurement point, and in a case where a point that is different from the first measurement point and the second measurement point and closer to the second measurement point than the first measurement point is specified, sets the point as the second corrected measurement point.

16. The measurement apparatus according to claim 1,
wherein the correction support information generation unit generates a second brightness profile on a second straight line passing through a third measurement point and a fourth measurement point specified for the ultrasound image and is orthogonal to the first straight line, and generates second correction support information for supporting correction of at least one position of the third measurement point or the fourth measurement point on the second straight line on the basis of the second brightness profile, and
wherein the measurement unit displays the second correction support information on the display unit, and on the basis of instructions input in a state where the second correction support information is displayed on the display unit, determines one of the third measurement point or a third corrected measurement point corrected from the third measurement point based on the second correction support information as a third final measurement point, determines one of the fourth measurement point or a fourth corrected measurement point corrected from the fourth measurement point based on the second correction support information as a fourth final measurement point, and measures an area of an elliptical measurement range passing through the first final measurement point, the second final measurement point, the third final measurement point, and the fourth final measurement point in the ultrasound image.

17. The measurement apparatus according to claim 16,
wherein the correction support information generation unit detects a third measurement candidate point that is a correction candidate of the third measurement point from a third range including the third measurement point in the second brightness profile on the basis of a brightness change amount of the third range, detects a fourth measurement candidate point that is a correction candidate of the fourth measurement point from a fourth range including the fourth measurement point in the second brightness profile on the basis of a brightness change amount of the fourth range, and generates information including one or both positions of the third measurement candidate point and the fourth measurement candidate point as the second correction support information, and further
wherein, in a state where the second straight line intersects points other than a midpoint of a line segment connecting the first final measurement point and the second final measurement point, the correction support information generation unit detects the third measurement candidate point and the fourth measurement candidate point at equal distances from the midpoint by moving the second straight line, the third measurement point, and the fourth measurement point in parallel along the line segment to have a state where the second straight line intersects the midpoint.

18. The ultrasound diagnostic apparatus comprising:
the measurement apparatus according to claim 1; and
an image processing unit that generates the ultrasound image based on an output signal of an ultrasonic endoscope.

19. A measurement method comprising:
a correction support information generation step of generating a first brightness profile on a first straight line passing through a first measurement point and a second measurement point specified for an ultrasound image generated by an ultrasound diagnostic apparatus and displayed on a display unit, and generating first correction support information for supporting correction of at least one position of the first measurement point or the second measurement point on the first straight line on the basis of the first brightness profile; and
a measurement step of displaying the first correction support information on the display unit, and on the basis of instructions input in a state where the first correction support information is displayed on the display unit, determining one of the first measurement point or a first corrected measurement point corrected from the first measurement point based on the first correction support information as a first final measurement point, determining one of the second measurement point or a second corrected measurement point corrected from the second measurement point based on the first correction support information as a second final measurement point, and measuring a size of a measurement range on the ultrasound image based on the first final measurement point and the second final measurement point.

20. A non-transitory computer readable recording medium storing a measurement program that causes a computer to perform:
a correction support information generation step of generating a first brightness profile on a first straight line passing through a first measurement point and a second measurement point specified for an ultrasound image generated by an ultrasound diagnostic apparatus and displayed on a display unit, and generating first correction support information for supporting correction of at least one position of the first measurement point or the second measurement point on the first straight line on the basis of the first brightness profile; and
a measurement step of displaying the first correction support information on the display unit, and on the basis of instructions input in a state where the first correction support information is displayed on the display unit, determining one of the first measurement point or a first corrected measurement point corrected from the first measurement point based on the first correction support information as a first final measurement point, determining one of the second measurement point or a second corrected measurement point corrected from the second measurement point based on the first correction support information as a second final measurement point, and measuring a size of a measurement range on the ultrasound image based on the first final measurement point and the second final measurement point.
